(19) United States
(12) Patent Application Publication
(10) Pub. No.: US 2024/0350118 A1
(43) Pub. Date: Oct. 24, 2024

(54) COREGISTRATION OF INTRALUMINAL DATA TO GUIDEWIRE IN EXTRALUMINAL IMAGE OBTAINED WITHOUT CONTRAST

(71) Applicant: PHILIPS IMAGE GUIDED THERAPY CORPORATION, SAN DIEGO, CA (US)

(72) Inventors: Rebecca Ann JENKINS, SAN DIEGO, CA (US); Ryan Michael SOTAK, COLORADO SPRINGS, CO (US); Ehud NACHTOMY, HERZLIYA (IL); Emily BROWN, AMSTERDAM (NL); Ronald Christiaan HELMSTRIJD, BEST (NL)

(21) Appl. No.: 18/290,174

(22) PCT Filed: May 10, 2022
(86) PCT No.: PCT/EP2022/062623

§ 371 Date: Nov. 10, 2023

Related U.S. Application Data
(60) Provisional application No. 63/187,983, filed on May 13, 2021.

(51) Int. Cl. A61B 8/00; A61B 6/00; A61B 6/12; A61B 6/46; A61B 8/12; A61B 90/00; A61M 25/09

(52) U.S. Cl. CPC A61B 8/4263; A61B 6/12; A61B 6/463; A61B 6/5247; A61B 8/12; A61B 2090/3966; A61M 2025/09166

(57) ABSTRACT

A co-registration system includes a processor circuit that co-registers intravascular data and user annotations to locations along a guidewire shown in an x-ray image. The processor circuit receives, from an x-ray imaging device, x-ray images of a blood vessel while anintravascular catheter moves through the blood vessel along a guidewire. The processor circuit receives, from the catheter, intravascular data representative of the blood vessel while the catheter moves through the blood vessel. The processor circuit co-registers the intravascular data to locations along the guidewire shown in the x-ray image received from the x-ray imaging device. The processor circuit also generates and co-registers annotations such as bookmarks to the guidewire.

### TECHNICAL FIELD

The present disclosure relates generally to coregistering intraluminal (e.g. intravascular data) with a guidewire of an extraluminal (e.g. x-ray) image obtained without contrast. In particular, intravascular data as well as user annotations may be displayed along a guidewire within an x-ray image without contrast.

### BACKGROUND

Physicians use many different medical diagnostic systems and tools to monitor a patient's health and diagnose and treat medical conditions. Different modalities of medical diagnostic systems may provide a physician with different images, models, and/or data relating to internal structures within a patient. These modalities include invasive devices and systems, such as intravascular systems, and non-invasive devices and systems, such as external ultrasound systems or x-ray systems. Using multiple diagnostic systems to examine a patient's anatomy provides a physician with added insight into the condition of the patient.

In the field of intravascular imaging and physiology measurement, co-registration of data from invasive devices (e.g. intravascular ultrasound (IVUS) devices) with images collected non-invasively (e.g. via x-ray angiography and/or x-ray venography) is a powerful technique for improving the efficiency and accuracy of vascular catheterization procedures. Co-registration identifies the locations of intravascular data measurements along a blood vessel by mapping the data to an x-ray image of the vessel. A physician may then see on an angiography image exactly where along the vessel a measurement was made, rather than estimate the location.

Coregistration of intravascular data to locations along a blood vessel typically requires introduction of a contrast agent into the patient vasculature. The contrast agent makes otherwise non-radiopaque blood vessels appear in x-ray images. When displayed to a user, the locations of the intravascular data are displayed along the contrast-filled filled vessel in the x-ray image. Introducing contrast agent, however, can be time consuming and prone to error. Some patients may also not tolerate contrast agent well, which can cause discomfort for the patient.

### SUMMARY

Embodiments of the present disclosure are systems, devices, and methods for coregistering intraluminal data and/or annotations to locations along a vessel identified by a guidewire in an extraluminal image. The vessel itself is not visible in the extraluminal image, such as an x-ray image. Rather, the guidewire that is positioned within the vessel is visible. The intravascular data and/or annotations are thus directly coregistered to locations along the guidewire, and only indirectly coregistered to locations along the vessel. For example, the intraluminal data can be intravascular imaging data, such as intravascular ultrasound (IVUS) images. The extraluminal image can be an x-ray fluoroscopy image acquired when no contrast agent is introduced to a patient's vasculature. A guidewire may be positioned within a blood vessel and an IVUS device may be moved along the guidewire as it acquires IVUS images of the vessel. While the IVUS device acquires images, an x-ray imaging system may acquire x-ray images of the same blood vessel. A processor circuit may receive these IVUS images and x-ray images and generate, based on the location of the IVUS device shown within the x-ray images, a pathway of the IVUS device through the patient anatomy. The processor circuit may then determine where along this path each IVUS images was acquired. This allows the user to easily locate exactly where an IVUS image was acquired along a vessel shown in an x-ray image. The guidewire along which the IVUS device travelled during the imaging procedure is constructed of a radiopaque material, so it is visible within the x-ray image without contrast.

In one aspect, because the pathway illustrating the movement of the IVUS device is of a similar shape to the guidewire, the IVUS images or other data that was coregistered to the pathway may similarly be coregistered to the guidewire itself seen within the x-ray image without contrast. In this way, the user may view the locations of acquired IVUS images along the guidewire without displaying the pathway or introducing contrast agent into the patient anatomy.

In another aspect, the user may identify IVUS images of interest with annotations such as bookmarks. The imaging system may also automatically identify IVUS images with bookmarks. These bookmarks may be displayed along a longitudinal view of the vessel (e.g., an ILD, such as an in-line digital image or image longitudinal display), the pathway generated by the system, or the guidewire within the x-ray image without contrast.

In an exemplary aspect, a system is provided. The system comprises a processor circuit configured for communication with an extraluminal imaging device and an intraluminal imaging catheter, wherein the processor circuit is configured to receive a plurality of extraluminal images obtained by the extraluminal imaging device during movement of an intraluminal imaging catheter along a guidewire within a body lumen of a patient, wherein the plurality of extraluminal images are obtained without a contrast agent within the body lumen, wherein the plurality of extraluminal images show the guidewire and a radiopaque portion of the intraluminal imaging catheter; receive a plurality of intraluminal images obtained by the intraluminal imaging catheter during the movement of the intraluminal imaging catheter; co-register the plurality of intraluminal images to corresponding positions along the guidewire based on the plurality of extraluminal images; and output, to a display in communication with the processor circuit, a screen display comprising an extraluminal image of the plurality of extraluminal images; an intraluminal image of the plurality of intraluminal images; and a first marking disposed along the guidewire in the extraluminal image at a corresponding position of the intraluminal image.

In one aspect, the processor circuit is configured to receive a user input selecting a different position along the guidewire in the extraluminal image; and modify the screen display to output the intraluminal image of the plurality of intraluminal images corresponding to the different position; and move the first marking to the different position along the guidewire in the extraluminal image. In one aspect, the screen display comprises a longitudinal view of the body lumen based on the plurality of intraluminal images; and a second marking at a site in the longitudinal view associated with the corresponding position of the intraluminal image; wherein the processor circuit is configured to receive a user input selecting a different site along the longitudinal view; and modify the screen display to move the first marking to a different position along the guidewire in the extraluminal imaging corresponding to the different site; output the intraluminal image of the plurality of intraluminal images corresponding to the different position along the guidewire in the extraluminal image; and move the second marking to the different site along the longitudinal view. In one aspect, at least a portion of the first marking is disposed over the guidewire in the extraluminal image. In one aspect, at least a portion of the first marking is spaced from the guidewire in the extraluminal image. In one aspect, the processor circuit is configured to determine a path of the movement based on corresponding locations of the radiopaque portion in the plurality of x-ray images, wherein a shape of the path matches a shape of the guidewire, and wherein the processor circuit is configured to co-register the plurality of intraluminal images to corresponding locations along the path, and wherein the processor circuit is configured to co-register the plurality of intraluminal images to the corresponding positions along the guidewire based on co-registering the plurality of IVUS images to the corresponding locations along the path. In one aspect, the screen display comprises a graphical representation of the path in the extraluminal image. In one aspect, the first marking is disposed along the graphical representation of the path in the extraluminal image. In one aspect, the screen display comprises a second marking in the extraluminal image representative of a beginning of the path; and a third marking in the extraluminal image representative of the end of the path. In one aspect, the screen display comprises a longitudinal view of the body lumen based on the plurality of intraluminal images, wherein the second marking corresponds to an initial intraluminal image of the plurality of intraluminal images, and wherein the third marking corresponds to a final intraluminal image of the plurality of intraluminal images. In one aspect, the screen display comprises a fourth marking disposed along the guidewire in the extraluminal image at the corresponding position of a bookmarked intraluminal image of the plurality of intraluminal images. In one aspect, the processor circuit is configured to automatically provide the fourth marking in the extraluminal image in response to the bookmarked intraluminal image being bookmarked. In one aspect, the bookmarked intraluminal image is manually bookmarked based on a user input received by the processor circuit. In one aspect, the bookmarked intraluminal image is automatically bookmarked by the processor circuit. In one aspect, the screen display comprises a longitudinal view of the body lumen based on the plurality of intraluminal images; and a fifth marking at a site in the longitudinal view associated with the corresponding position of the bookmarked intraluminal image.

In an exemplary, a system is provided. The system comprises an intravascular ultrasound (IVUS) imaging catheter; and a processor circuit configured for communication with an x-ray imaging device and the IVUS imaging catheter, wherein the processor circuit is configured to receive a plurality of x-ray images obtained by the x-ray imaging device during movement of the IVUS imaging catheter along a guidewire within a blood vessel of a patient, wherein the plurality of x-ray images are obtained without a contrast agent within the blood vessel, wherein the plurality of x-ray images show the guidewire and a radiopaque portion of the IVUS imaging catheter; receive a plurality of IVUS images obtained by the IVUS imaging catheter during the movement of the IVUS imaging catheter; determine a path of the movement based on corresponding locations of the radiopaque portion in the plurality of x-ray images, wherein a shape of the path matches a shape of the guidewire; co-register the plurality of IVUS images to corresponding locations along the path such that the plurality of IVUS images are co-registered with corresponding positions along the guidewire; and output, to a display in communication with the processor circuit, a screen display comprising an x-ray image of the plurality of x-ray images; an IVUS image of the plurality of IVUS images; and a first marking along the guidewire in the x-ray image representative a corresponding position of the IVUS image.

In one aspect, the screen display further comprises a second marking along the guidewire in the IVUS image representative of a bookmarked IVUS image of the plurality of IVUS images.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a schematic diagram of an intraluminal imaging and x-ray system, according to aspects of the present disclosure.

FIG. 2 is a diagrammatic top view of an ultrasound imaging assembly in a flat configuration, according to aspects of the present disclosure.

FIG. 3 is a diagrammatic perspective view of the ultrasound imaging assembly shown in FIG. 2 in a rolled configuration around a support member, according to aspects of the present disclosure.

FIG. 4 is a diagrammatic cross-sectional side view of the ultrasound imaging assembly shown in FIG. 3, according to aspects of the present disclosure.

FIG. 5 is a schematic diagram of a processor circuit, according to aspects of the present disclosure.

FIG. 6 is a diagrammatic view of an x-ray fluoroscopy image illustrating a pullback procedure, according to aspects of the present disclosure.

FIG. 7 is a diagrammatic view of a relationship between x-ray fluoroscopy images, intravascular data, and a path defined by the motion of an intravascular device, according to aspects of the present disclosure.

FIG. 8A is a diagrammatic view of an x-ray image that does not include contrast inside the vessel and does include a guidewire positioned within the vessel, according to aspects of the present disclosure.

FIG. 8B is a diagrammatic view of the x-ray image of FIG. 8B identifying the vessel in which the guidewire is positioned.

FIG. 9 is a diagrammatic view of a graphical user interface displaying an intravascular image coregistered to an x-ray image, according to aspects of the present disclosure.

FIG. 10 is a diagrammatic view of a graphical user interface displaying an intravascular image coregistered to an x-ray image, according to aspects of the present disclosure.

FIG. 11 is a flow diagram for a method for co-registering intravascular data and/or annotations to locations along a guidewire within an x-ray image obtained without contrast, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

The devices, systems, and methods described herein can include one or more features described in U.S. Provisional Application No. 63/87,962, filed May 13, 2021, and titled “Coregistration Reliability with Extraluminal Image and Intraluminal Data” (Atty Dkt No. 2021PF00090/44755.2198PV01), U.S. Provisional Application No. 63/187,964, filed May 13, 2021, and titled “Pathway Modification for Coregistration of Extraluminal Image and Intraluminal Data” (Atty Dkt No. 2021PF00091/44755.2199PV01), U.S. Provisional Application No. 63/187,990, filed May 3, 2021, and titled “Preview of Intraluminal Ultrasound Image Along Longitudinal View of Body Lumen” (Atty Dkt No. 2021PF00093/44755.2201PV01), and U.S. Provisional Application No. 63/187,961, filed May 13, 2021, and titled “Intraluminal Treatment Guidance from Prior Extraluminal Imaging, Intraluminal Data, and Coregistration” (Atty Dkt No. 2021PF00012/44755.2192PV01), each of which is incorporated by reference herein in its entirety.

The devices, systems, and methods described herein can also include one or more features described in European Application No. 21154591.8, filed Feb. 1, 2021, and titled “X-Ray and Intravascular Ultrasound Image Registration”, which is incorporated by reference herein in its entirety.

The devices, systems, and methods described herein can also include one or more features described in U.S. Publication No. 2020/0129144, titled “Disease Specific and Treatment Type Specific Control of Intraluminal Ultrasound Imaging”, U.S. Publication No. 2020/0129142, titled “Intraluminal Ultrasound Navigation Guidance and Associated Devices, Systems, And Methods”, U.S. Publication No. 2020/0129148, titled “Intraluminal Ultrasound Imaging with Automatic and Assisted Labels And Bookmarks”, U.S. Publication No. 2020/0129158, titled “Graphical Longitudinal Display for Intraluminal Ultrasound Imaging and Associated Devices, Systems, and Methods”, U.S. Publication No. 2020/0129147, titled “Intraluminal Ultrasound Vessel Border Selection and Associated Devices, Systems, and Methods”, U.S. Publication No. 2020/0129159, titled “Intraluminal Ultrasound Directional Guidance and Associated Devices, Systems, and Methods”, U.S. Publication No. 2020/0129143, titled “Speed Determination for Intraluminal Ultrasound Imaging and Associated Devices, Systems, And Methods”, each of which is incorporated by reference herein in its entirety.

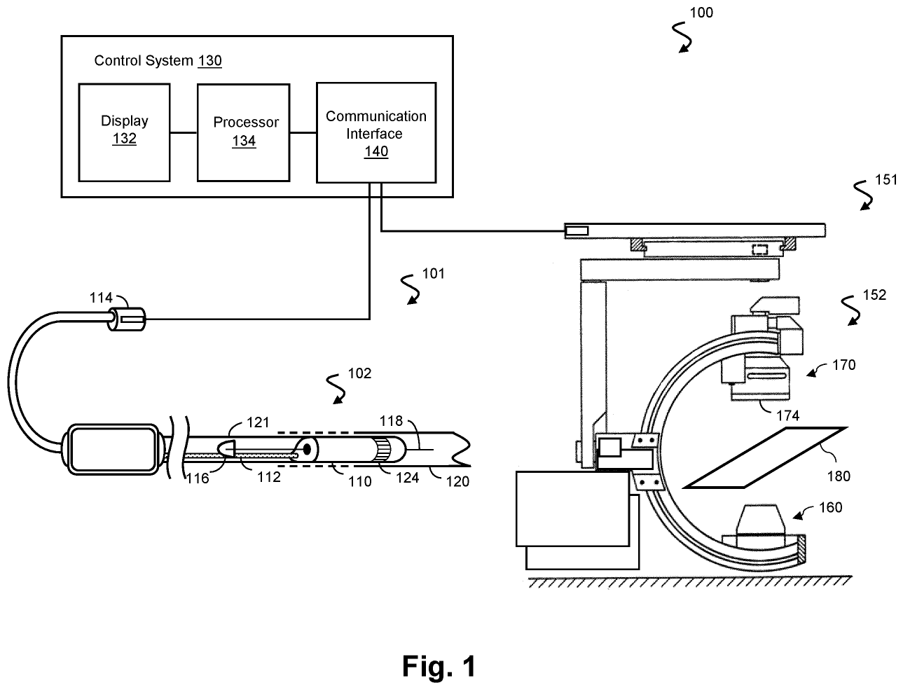

FIG. 1 is a schematic diagram of an intraluminal imaging and x-ray system 100, according to aspects of the present disclosure. In some embodiments, the intraluminal imaging and x-ray system 100 may include two separate systems or be a combination of two systems: an intraluminal sensing system 101 and an extraluminal imaging system 151. The intraluminal sensing system 101 obtains medical data about a patient's body while the intraluminal device 102 is positioned inside the patient's body. For example, the intraluminal sensing system 101 can control the intraluminal device 102 to obtain intraluminal images of the inside of the patient's body while the intraluminal device 102 is inside the patient's body. The extraluminal imaging system 151 obtains medical data about the patient's body while the extraluminal imaging device 152 is positioned outside the patient's body. For example, the extraluminal imaging system 151 can control extraluminal imaging device 152 to obtain extraluminal images of the inside of the patient's body while the extraluminal imaging device 152 is outside the patient's body.

The intraluminal imaging system 101 may be in communication with the extraluminal imaging system 151 through any suitable components. Such communication may be established through a wired cable, through a wireless signal, or by any other means. In addition, the intraluminal imaging system 101 may be in continuous communication with the x-ray system 151 or may be in intermittent communication. For example, the two systems may be brought into temporary communication via a wired cable, or brought into communication via a wireless communication, or through any other suitable means at some point before, after, or during an examination. In addition, the intraluminal system 101 may receive data such as x-ray images, annotated x-ray images, metrics calculated with the x-ray imaging system 151, information regarding dates and times of examinations, types and/or severity of patient conditions or diagnoses, patient history or other patient information, or any suitable data or information from the x-ray imaging system 151. The x-ray imaging system 151 may also receive any of these data from the intraluminal imaging system 101. In some embodiments, and as shown in FIG. 1, the intraluminal imaging system 101 and the x-ray imaging system 151 may be in communication with the same control system 130. In this embodiment, both systems may be in communication with the same display 132, processor 134, and communication interface 140 shown as well as in communication with any other components implemented within the control system 130.

In some embodiments, the system 100 may not include a control system 130 in communication with the intraluminal imaging system 101 and the x-ray imaging system 151. Instead, the system 100 may include two separate control systems. For example, one control system may be in communication with or be a part of the intraluminal imaging system 101 and an additional separate control system may be in communication with or be a part of the x-ray imaging system 151. In this embodiment, the separate control systems of both the intraluminal imaging system 101 and the x-ray imaging system 151 may be similar to the control system 130. For example, each control system may include various components or systems such as a communication interface, processor, and/or a display. In this embodiment, the control system of the intraluminal imaging system 101 may perform any or all of the coregistration steps described in the present disclosure. Alternatively, the control system of the x-ray imaging system 151 may perform the coregistration steps described.

The intraluminal imaging system 101 can be an ultrasound imaging system. In some instances, the intraluminal imaging system 101 can be an intravascular ultrasound (IVUS) imaging system. The intraluminal imaging system 101 may include an intraluminal imaging device 102, such as a catheter, guide wire, or guide catheter, in communication with the control system 130. The control system 130 may include a display 132, a processor 134, and a communication interface 140 among other components. The intraluminal imaging device 102 can be an ultrasound imaging device. In some instances, the device 102 can be an IVUS imaging device, such as a solid-state IVUS device.

At a high level, the IVUS device 102 emits ultrasonic energy from a transducer array 124 included in a scanner assembly, also referred to as an IVUS imaging assembly, mounted near a distal end of the catheter device. The ultrasonic energy is reflected by tissue structures in the surrounding medium, such as a vessel 120, or another body lumen surrounding the scanner assembly 110, and the ultrasound echo signals are received by the transducer array 124. In that regard, the device 102 can be sized, shaped, or otherwise configured to be positioned within the body lumen of a patient. The communication interface 140 transfers the received echo signals to the processor 134 of the control system 130 where the ultrasound image (including flow information in some embodiments) is reconstructed and displayed on the display 132. The control system 130, including the processor 134, can be operable to facilitate the features of the IVUS imaging system 101 described herein. For example, the processor 134 can execute computer readable instructions stored on the non-transitory tangible computer readable medium.

The communication interface 140 facilitates communication of signals between the control system 130 and the scanner assembly 110 included in the IVUS device 102. This communication includes the steps of. (1) providing commands to integrated circuit controller chip(s) included in the scanner assembly 110 to select the particular transducer array element(s), or acoustic element(s), to be used for transmit and receive, (2) providing the transmit trigger signals to the integrated circuit controller chip(s) included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s) of the scanner assembly 110. In some embodiments, the communication interface 140 performs preliminary processing of the echo data prior to relaying the data to the processor 134. In examples of such embodiments, the communication interface 140 performs amplification, filtering, and/or aggregating of the data. In an embodiment, the communication interface 140 also supplies high- and low-voltage DC power to support operation of the device 102 including circuitry within the scanner assembly 110.

The processor 134 receives the echo data from the scanner assembly 110 by way of the communication interface 140 and processes the data to reconstruct an image of the tissue structures in the medium surrounding the scanner assembly 110. The processor 134 outputs image data such that an image of the lumen 120, such as a cross-sectional image of the vessel 120, is displayed on the display 132. The lumen 120 may represent fluid filled or surrounded structures, both natural and man-made. The lumen 120 may be within a body of a patient. The lumen 120 may be a blood vessel, such as an artery or a vein of a patient's vascular system, including cardiac vasculature, peripheral vasculature, neural vasculature, renal vasculature, and/or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be used to examine man-made structures such as, but without limitation, heart valves, stents, shunts, filters and other devices.

In some embodiments, the IVUS device includes some features similar to traditional solid-state IVUS catheters, such as the EagleEye® catheter, Visions PV 0.014P RX catheter, Visions PV 0.018 catheter, Visions PV 0.035, and Pioneer Plus catheter, each of which are available from Koninklijke Philips N.V, and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors. It is understood that any suitable gauge wire can be used for the conductors. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a patient interface module (PIM) connector 114 at a proximal end of the device 102. The PIM connector 114 electrically couples the transmission line bundle 112 to the communication interface 140 and physically couples the IVUS device 102 to the communication interface 140. In some embodiments, the communication interface 140 may be a PIM. In an embodiment, the IVUS device 102 further includes a guide wire exit port 116. Accordingly, in some instances the IVUS device 102 is a rapid-exchange catheter. The guide wire exit port 116 allows a guide wire 118 to be inserted towards the distal end to direct the device 102 through the vessel 120.

In some embodiments, the intraluminal imaging device 102 may acquire intravascular images of any suitable imaging modality, including optical coherence tomography (OCT) and intravascular photoacoustic (IVPA).

In some embodiments, the intraluminal device 102 is a pressure sensing device (e.g., pressure-sensing guidewire) that obtains intraluminal (e.g., intravascular) pressure data, and the intraluminal system 101 is an intravascular pressure sensing system that determines pressure ratios based on the pressure data, such as fractional flow reserve (FFR), instantaneous wave-free ratio (iFR), and/or other suitable ratio between distal pressure and proximal/aortic pressure (Pd/Pa). In some embodiments, the intraluminal device 102 is a flow sensing device (e.g., flow-sensing guidewire) that obtains intraluminal (e.g., intravascular) flow data, and the intraluminal system 101 is an intravascular flow sensing system that determines flow-related values based on the pressure data, such as coronary flow reserve (CFR), flow velocity, flow volume, etc.

The x-ray imaging system 151 may include an x-ray imaging apparatus or device 152 configured to perform x-ray imaging, angiography, fluoroscopy, radiography, venography, among other imaging techniques. The x-ray imaging system 151 can generate a single x-ray image (e.g., an angiogram or venogram) or multiple (e.g., two or more) x-ray images (e.g., a video and/or fluoroscopic image stream) based on x-ray image data collected by the x-ray device 152. The x-ray imaging device 152 may be of any suitable type, for example, it may be a stationary x-ray system such as a fixed c-arm x-ray device, a mobile c-arm x-ray device, a straight arm x-ray device, or a u-arm device. The x-ray imaging device 152 may additionally be any suitable mobile device. The x-ray imaging device 152 may also be in communication with the control system 130. In some embodiments, the x-ray system 151 may include a digital radiography device or any other suitable device.

The x-ray device 152 as shown in FIG. 1 includes an x-ray source 160 and an x-ray detector 170 including an input screen 174. The x-ray source 160 and the detector 170 may be mounted at a mutual distance. Positioned between the x-ray source 160 and the x-ray detector 170 may be an anatomy of a patient or object 180. For example, the anatomy of the patient (including the vessel 120) can be positioned between the x-ray source 160 and the x-ray detector 170.

The x-ray source 160 may include an x-ray tube adapted to generate x-rays. Some aspects of the x-ray source 160 may include one or more vacuum tubes including a cathode in connection with a negative lead of a high-voltage power source and an anode in connection with a positive lead of the same power source. The cathode of the x-ray source 160 may additionally include a filament. The filament may be of any suitable type or constructed of any suitable material, including tungsten or rhenium tungsten, and may be positioned within a recessed region of the cathode. One function of the cathode may be to expel electrons from the high voltage power source and focus them into a well-defined beam aimed at the anode. The anode may also be constructed of any suitable material and may be configured to create x-radiation from the emitted electrons of the cathode. In addition, the anode may dissipate heat created in the process of generating x-radiation. The anode may be shaped as a beveled disk and, in some embodiments, may be rotated via an electric motor. The cathode and anode of the x-ray source 160 may be housed in an airtight enclosure, sometimes referred to as an envelope.

In some embodiments, the x-ray source 160 may include a radiation object focus which influences the visibility of an image. The radiation object focus may be selected by a user of the system 100 or by a manufacture of the system 100 based on characteristics such as blurring, visibility, heat-dissipating capacity, or other characteristics. In some embodiments, an operator or user of the system 100 may switch between different provided radiation object foci in a point-of-care setting.

The detector 170 may be configured to acquire x-ray images and may include the input screen 174. The input screen 174 may include one or more intensifying screens configured to absorb x-ray energy and convert the energy to light. The light may in turn expose a film. The input screen 174 may be used to convert x-ray energy to light in embodiments in which the film may be more sensitive to light than x-radiation. Different types of intensifying screens within the image intensifier may be selected depending on the region of a patient to be imaged, requirements for image detail and/or patient exposure, or any other factors. Intensifying screens may be constructed of any suitable materials, including barium lead sulfate, barium strontium sulfate, barium fluorochloride, yttrium oxysulfide, or any other suitable material. The input screen 374 may be a fluorescent screen or a film positioned directly adjacent to a fluorescent screen. In some embodiments, the input screen 374 may also include a protective screen to shield circuitry or components within the detector 370 from the surrounding environment. In some embodiments, the x-ray detector 170 may include a flat panel detector (FPD). The detector 170 may be an indirect conversion FPD or a direct conversion FPD. The detector 170 may also include charge-coupled devices (CCDs). The x-ray detector 370 may additionally be referred to as an x-ray sensor.

The object 180 may be any suitable object to be imaged. In an exemplary embodiment, the object may be the anatomy of a patient. More specifically, the anatomy to be imaged may include chest, abdomen, the pelvic region, neck, legs, head, feet, a region with cardiac vasculature, or a region containing the peripheral vasculature of a patient and may include various anatomical structures such as, but not limited to, organs, tissue, blood vessels and blood, gases, or any other anatomical structures or objects. In other embodiments, the object may be or include man-made structures.

In some embodiments, the x-ray imaging system 151 may be configured to obtain x-ray images without contrast. In some embodiments, the x-ray imaging system 151 may be configured to obtain x-ray images with contrast (e.g., angiogram or venogram). In such embodiments, a contrast agent or x-ray dye may be introduced to a patient's anatomy before imaging. The contrast agent may also be referred to as a radiocontrast agent, contrast material, contrast dye, or contrast media. The contrast dye may be of any suitable material, chemical, or compound and may be a liquid, powder, paste, tablet, or of any other suitable form. For example, the contrast dye may be iodine-based compounds, barium sulfate compounds, gadolinium-based compounds, or any other suitable compounds. The contrast agent may be used to enhance the visibility of internal fluids or structures within a patient's anatomy. The contrast agent may absorb external x-rays, resulting in decreased exposure on the x-ray detector 170.

In some embodiments, the extraluminal imaging system 151 could be any suitable extraluminal imaging device, such as computed tomography (CT) or magnetic resonance imaging (MRI).

When the control system 130 is in communication with the x-ray system 151, the communication interface 140 facilitates communication of signals between the control system 130 and the x-ray device 152. This communication includes providing control commands to the x-ray source 160 and/or the x-ray detector 170 of the x-ray device 152 and receiving data from the x-ray device 152. In some embodiments, the communication interface 140 performs preliminary processing of the x-ray data prior to relaying the data to the processor 134. In examples of such embodiments, the communication interface 140 may perform amplification, filtering, and/or aggregating of the data. In an embodiment, the communication interface 140 also supplies high- and low-voltage DC power to support operation of the device 152 including circuitry within the device.

The processor 134 receives the x-ray data from the x-ray device 152 by way of the communication interface 140 and processes the data to reconstruct an image of the anatomy being imaged. The processor 134 outputs image data such that an image is displayed on the display 132. In an embodiment in which the contrast agent is introduced to the anatomy of a patient and a venogram is to be generated, the particular areas of interest to be imaged may be one or more blood vessels or other section or part of the human vasculature. The contrast agent may identify fluid filled structures, both natural and/or man-made, such as arteries or veins of a patient's vascular system, including cardiac vasculature, peripheral vasculature, neural vasculature, renal vasculature, and/or any other suitable lumen inside the body. For example, the x-ray device 152 may be used to examine any number of anatomical locations and tissue types, including without limitation all the organs, fluids, or other structures or parts of an anatomy previously mentioned. In addition to natural structures, the x-ray device 152 may be used to examine man-made structures such as any of the previously mentioned structures.

The processor 134 may be configured to receive an x-ray image that was stored by the x-ray imaging device 152 during a clinical procedure. The images may be further enhanced by other information such as patient history, patient record, IVUS imaging, pre-operative ultrasound imaging, pre-operative CT, or any other suitable data.

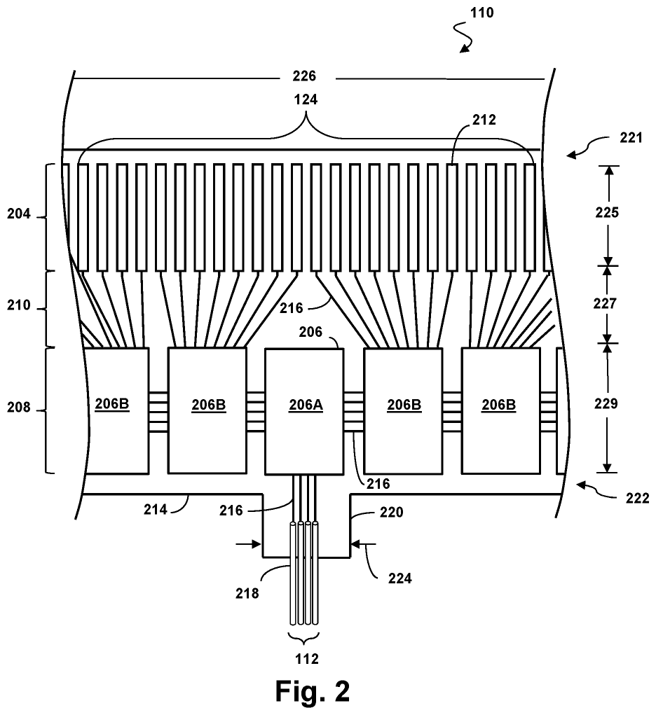

FIG. 2 is a diagrammatic top view of a portion of a flexible assembly 110, according to aspects of the present disclosure. The flexible assembly 110 includes a transducer array 124 formed in a transducer region 204 and transducer control logic dies 206 (including dies 206A and 206B) formed in a control region 208, with a transition region 210 disposed therebetween. The transducer array 124 includes an array of ultrasound transducer elements 212. The transducer control logic dies 206 are mounted on a flexible substrate 214 into which the transducer elements 212 have been previously integrated. The flexible substrate 214 is shown in a flat configuration in FIG. 2. Though six control logic dies 206 are shown in FIG. 2, any number of control logic dies 206 may be used. For example, one, two, three, four, five, six, seven, eight, nine, ten, or more control logic dies 206 may be used.

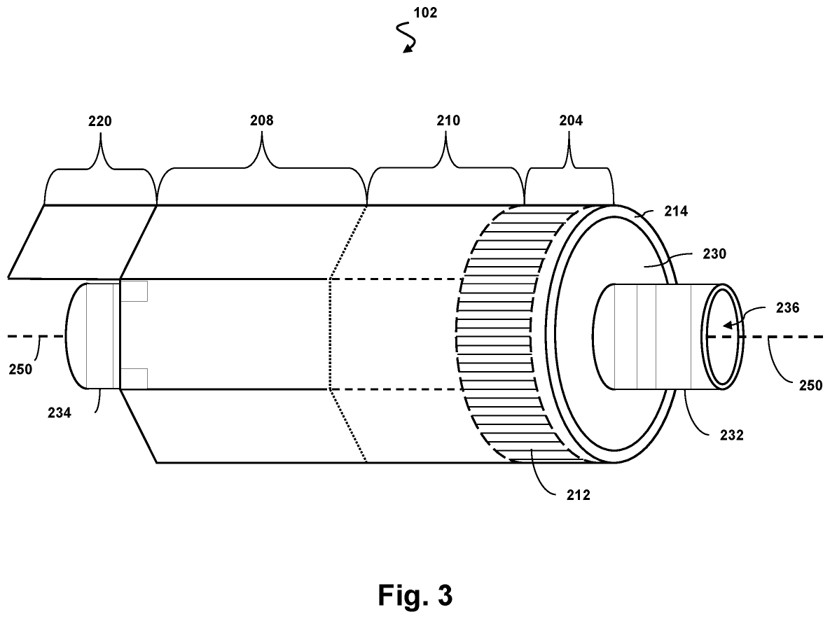

The flexible substrate 214, on which the transducer control logic dies 206 and the transducer elements 212 are mounted, provides structural support and interconnects for electrical coupling. The flexible substrate 214 may be constructed to include a film layer of a flexible polyimide material such as KAPTON™ (trademark of DuPont). Other suitable materials include polyester films, polyimide films, polyethylene napthalate films, or polyetherimide films, liquid crystal polymer, other flexible printed semiconductor substrates as well as products such as Upilex® (registered trademark of Ube Industries) and TEFLON® (registered trademark of E.I. du Pont). In the flat configuration illustrated in FIG. 2, the flexible substrate 214 has a generally rectangular shape. As shown and described herein, the flexible substrate 214 is configured to be wrapped around a support member 230 (FIG. 3) in some instances. Therefore, the thickness of the film layer of the flexible substrate 214 is generally related to the degree of curvature in the final assembled flexible assembly 110. In some embodiments, the film layer is between 5 μm and 100 μm, with some particular embodiments being between 5 μm and 25.1 μm, e.g., 6 μm.

The set of transducer control logic dies 206 is a non-limiting example of a control circuit. The transducer region 204 is disposed at a distal portion 221 of the flexible substrate 214. The control region 208 is disposed at a proximal portion 222 of the flexible substrate 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or, the length 227 of the transition region 210 may be less than lengths 225 and 229, the length 227 of the transition region 210 can be greater than lengths 225, 229 of the transducer region and controller region, respectively.

The control logic dies 206 are not necessarily homogenous. In some embodiments, a single controller is designated a master control logic die 206A and contains the communication interface for cable 112, between a processing system, e.g., processing system 106, and the flexible assembly 110. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a plurality of transducer elements 512 positioned on a transducer element 212 to emit an ultrasonic signal and selects a transducer element 212 to receive an echo. In the depicted embodiment, the master controller 206A does not directly control any transducer elements 212. In other embodiments, the master controller 206A drives the same number of transducer elements 212 as the slave controllers 206B or drives a reduced set of transducer elements 212 as compared to the slave controllers 206B. In an exemplary embodiment, a single master controller 206A and eight slave controllers 206B are provided with eight transducers assigned to each slave controller 206B.

To electrically interconnect the control logic dies 206 and the transducer elements 212, in an embodiment, the flexible substrate 214 includes conductive traces 216 formed in the film layer that carry signals between the control logic dies 206 and the transducer elements 212. In particular, the conductive traces 216 providing communication between the control logic dies 206 and the transducer elements 212 extend along the flexible substrate 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flexible substrate 214. Suitable materials for the conductive traces 216 include copper, gold, aluminum, silver, tantalum, nickel, and tin, and may be deposited on the flexible substrate 214 by processes such as sputtering, plating, and etching. In an embodiment, the flexible substrate 214 includes a chromium adhesion layer. The width and thickness of the conductive traces 216 are selected to provide proper conductivity and resilience when the flexible substrate 214 is rolled. In that regard, an exemplary range for the thickness of a conductive trace 216 and/or conductive pad is between 1-5 μm. For example, in an embodiment, 5 μm conductive traces 216 are separated by 5 μm of space. The width of a conductive trace 216 on the flexible substrate may be further determined by the width of the conductor 218 to be coupled to the trace or pad.

The flexible substrate 214 can include a conductor interface 220 in some embodiments. The conductor interface 220 can be in a location of the flexible substrate 214 where the conductors 218 of the cable 112 are coupled to the flexible substrate 214. For example, the bare conductors of the cable 112 are electrically coupled to the flexible substrate 214 at the conductor interface 220. The conductor interface 220 can be tab extending from the main body of flexible substrate 214. In that regard, the main body of the flexible substrate 214 can refer collectively to the transducer region 204, controller region 208, and the transition region 210. In the illustrated embodiment, the conductor interface 220 extends from the proximal portion 222 of the flexible substrate 214. In other embodiments, the conductor interface 220 is positioned at other parts of the flexible substrate 214, such as the distal portion 221, or the flexible substrate 214 may lack the conductor interface 220. A value of a dimension of the tab or conductor interface 220, such as a width 224, can be less than the value of a dimension of the main body of the flexible substrate 214, such as a width 226. In some embodiments, the substrate forming the conductor interface 220 is made of the same material(s) and/or is similarly flexible as the flexible substrate 214. In other embodiments, the conductor interface 220 is made of different materials and/or is comparatively more rigid than the flexible substrate 214. For example, the conductor interface 220 can be made of a plastic, thermoplastic, polymer, hard polymer, etc., including polyoxymethylene (e.g., DELRIN®), polyether ether ketone (PEEK), nylon, Liquid Crystal Polymer (LCP), and/or other suitable materials.

FIG. 3 illustrates a perspective view of the scanner assembly 110 in a rolled configuration. In some instances, the flexible substrate 214 is transitioned from a flat configuration (FIG. 2) to a rolled or more cylindrical configuration (FIG. 3). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND SENSING ASSEMBLY HAVING A FLEXIBLE SUBSTRATE,” each of which is hereby incorporated by reference in its entirety.

Depending on the application and embodiment of the presently disclosed invention, transducer elements 212 may be piezoelectric transducers, single crystal transducer, or PZT (lead zirconate titanate) transducers. In other embodiments, the transducer elements of transducer array 124 may be flexural transducers, piezoelectric micromachined ultrasonic transducers (PMUTs), capacitive micromachined ultrasonic transducers (CMUTs), or any other suitable type of transducer element. In such embodiments, transducer elements 212 may comprise an elongate semiconductor material or other suitable material that allows micromachining or similar methods of disposing extremely small elements or circuitry on a substrate.

In some embodiments, the transducer elements 212 and the controllers 206 can be positioned in an annular configuration, such as a circular configuration or in a polygon configuration, around a longitudinal axis 250 of a support member 230. It is understood that the longitudinal axis 250 of the support member 230 may also be referred to as the longitudinal axis of the scanner assembly 110, the flexible elongate member 121, or the device 102. For example, a cross-sectional profile of the imaging assembly 110 at the transducer elements 212 and/or the controllers 206 can be a circle or a polygon. Any suitable annular polygon shape can be implemented, such as one based on the number of controllers or transducers, flexibility of the controllers or transducers, etc. Some examples may include a pentagon, hexagon, heptagon, octagon, nonagon, decagon, etc. In some examples, the transducer controllers 206 may be used for controlling the ultrasound transducers 512 of transducer elements 212 to obtain imaging data associated with the vessel 120.

The support member 230 can be referenced as a unibody in some instances. The support member 230 can be composed of a metallic material, such as stainless steel, or a non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. In some embodiments, support member 230 may be composed of 303 stainless steel. The support member 230 can be a ferrule having a distal flange or portion 232 and a proximal flange or portion 234. The support member 230 can be tubular in shape and define a lumen 236 extending longitudinally therethrough. The lumen 236 can be sized and shaped to receive the guide wire 118. The support member 230 can be manufactured using any suitable process. For example, the support member 230 can be machined and/or electrochemically machined or laser milled, such as by removing material from a blank to shape the support member 230, or molded, such as by an injection molding process or a micro injection molding process.

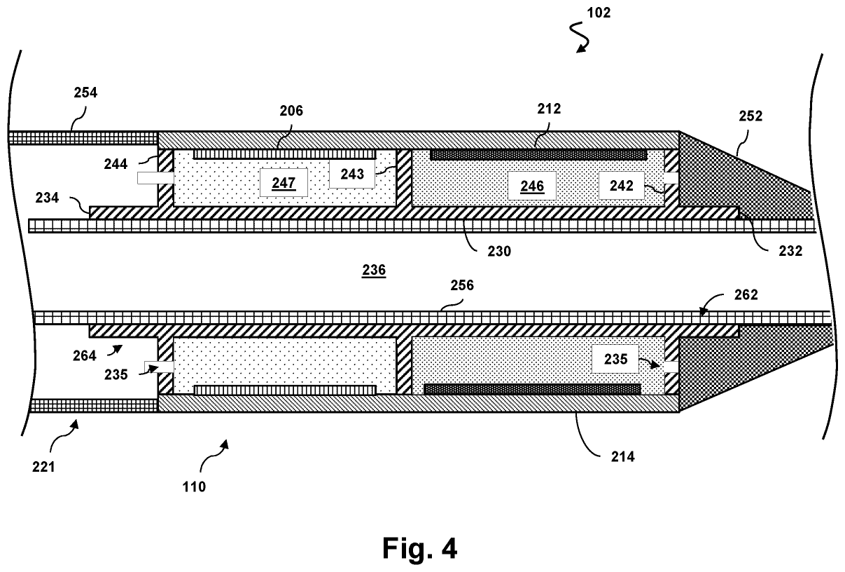

Referring now to FIG. 4, shown therein is a diagrammatic cross-sectional side view of a distal portion of the intraluminal imaging device 102, including the flexible substrate 214 and the support member 230, according to aspects of the present disclosure. The lumen 236 may be connected with the entry/exit port 116 and is sized and shaped to receive the guide wire 118 (FIG. 1). In some embodiments, the support member 230 may be integrally formed as a unitary structure, while in other embodiments the support member 230 may be formed of different components, such as a ferrule and stands 242, 243, and 244, that are fixedly coupled to one another. In some cases, the support member 230 and/or one or more components thereof may be completely integrated with inner member 256. In some cases, the inner member 256 and the support member 230 may be joined as one, e.g., in the case of a polymer support member.

Stands 242, 243, and 244 that extend vertically are provided at the distal, central, and proximal portions respectively, of the support member 230. The stands 242, 243, and 244 elevate and support the distal, central, and proximal portions of the flexible substrate 214. In that regard, portions of the flexible substrate 214, such as the transducer portion 204 (or transducer region 204), can be spaced from a central body portion of the support member 230 extending between the stands 242, 243, and 244. The stands 242, 243, 244 can have the same outer diameter or different outer diameters. For example, the distal stand 242 can have a larger or smaller outer diameter than the central stand 243 and/or proximal stand 244 and can also have special features for rotational alignment as well as control chip placement and connection.

To improve acoustic performance, the cavity between the transducer array 212 and the surface of the support member 230 may be filled with an acoustic backing material 246. The liquid backing material 246 can be introduced between the flexible substrate 214 and the support member 230 via passageway 235 in the stand 242, or through additional recesses as will be discussed in more detail hereafter. The backing material 246 may serve to attenuate ultrasound energy emitted by the transducer array 212 that propagates in the undesired, inward direction.

The cavity between the circuit controller chips 206 and the surface of the support member 230 may be filled with an underfill material 247. The underfill material 247 may be an adhesive material (e.g. an epoxy) which provides structural support for the circuit controller chips 206 and/or the flexible substrate 214. The underfill 247 may additionally be any suitable material.

In some embodiments, the central body portion of the support member can include recesses allowing fluid communication between the lumen of the unibody and the cavities between the flexible substrate 214 and the support member 230. Acoustic backing material 246 and/or underfill material 247 can be introduced via the cavities (during an assembly process, prior to the inner member 256 extending through the lumen of the unibody. In some embodiments, suction can be applied via the passageways 235 of one of the stands 242, 244, or to any other suitable recess while the liquid backing material 246 is fed between the flexible substrate 214 and the support member 230 via the passageways 235 of the other of the stands 242, 244, or any other suitable recess. The backing material can be cured to allow it to solidify and set. In various embodiments, the support member 230 includes more than three stands 242, 243, and 244, only one or two of the stands 242, 243, 244, or none of the stands. In that regard the support member 230 can have an increased diameter distal portion 262 and/or increased diameter proximal portion 264 that is sized and shaped to elevate and support the distal and/or proximal portions of the flexible substrate 214.

The support member 230 can be substantially cylindrical in some embodiments. Other shapes of the support member 230 are also contemplated including geometrical, non-geometrical, symmetrical, non-symmetrical, cross-sectional profiles. As the term is used herein, the shape of the support member 230 may reference a cross-sectional profile of the support member 230. Different portions of the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can comprise a flexible elongate member. The proximal inner member 256 can be received within a proximal flange 234. The proximal outer member 254 abuts and is in contact with the proximal end of flexible substrate 214. A distal tip member 252 is coupled to the distal portion 262 of the support member 230. For example, the distal member 252 is positioned around the distal flange 232. The tip member 252 can abut and be in contact with the distal end of flexible substrate 214 and the stand 242. In other embodiments, the proximal end of the tip member 252 may be received within the distal end of the flexible substrate 214 in its rolled configuration. In some embodiments there may be a gap between the flexible substrate 214 and the tip member 252. The distal member 252 can be the distal-most component of the intraluminal imaging device 102. The distal tip member 252 may be a flexible, polymeric component that defines the distal-most end of the imaging device 102. The distal tip member 252 may additionally define a lumen in communication with the lumen 236 defined by support member 230. The guide wire 118 may extend through lumen 236 as well as the lumen defined by the tip member 252.

One or more adhesives can be disposed between various components at the distal portion of the intraluminal imaging device 102. For example, one or more of the flexible substrate 214, the support member 230, the distal member 252, the proximal inner member 256, the transducer array 212, and/or the proximal outer member 254 can be coupled to one another via an adhesive. Stated differently, the adhesive can be in contact with e.g. the transducer array 212, the flexible substrate 214, the support member 230, the distal member 252, the proximal inner member 256, and/or the proximal outer member 254, among other components.

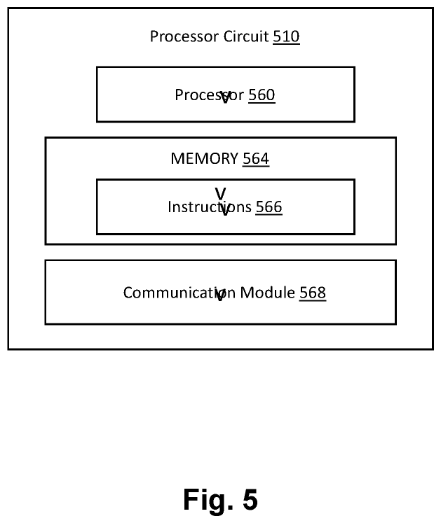

FIG. 5 is a schematic diagram of a processor circuit, according to aspects of the present disclosure. The processor circuit 510 may be implemented in the control system 130 of FIG. 1, the intraluminal imaging system 101, and/or the x-ray imaging system 151, or any other suitable location. In an example, the processor circuit 510 may be in communication with intraluminal imaging device 102, the x-ray imaging device 152, the display 132 within the system 100. The processor circuit 510 may include the processor 134 and/or the communication interface 140 (FIG. 1). One or more processor circuits 510 are configured to execute the operations described herein. As shown, the processor circuit 510 may include a processor 560, a memory 564, and a communication module 568. These elements may be in direct or indirect communication with each other, for example via one or more buses.

The processor 560 may include a CPU, a GPU, a DSP, an application-specific integrated circuit (ASIC), a controller, an FPGA, another hardware device, a firmware device, or any combination thereof configured to perform the operations described herein. The processor 560 may also be implemented as a combination of computing devices, e.g., a combination of a DSP and a microprocessor, a plurality of microprocessors, one or more microprocessors in conjunction with a DSP core, or any other such configuration.

The memory 564 may include a cache memory (e.g., a cache memory of the processor 560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 564 includes a non-transitory computer-readable medium. The memory 564 may store instructions 566. The instructions 566 may include instructions that, when executed by the processor 560, cause the processor 560 to perform the operations described herein with reference to the probe 110 and/or the host 130 (FIG. 1). Instructions 566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 568 can include any electronic circuitry and/or logic circuitry to facilitate direct or indirect communication of data between the processor circuit 510, the probe 110, and/or the display 132 and/or display 132. In that regard, the communication module 568 can be an input/output (I/O) device. In some instances, the communication module 568 facilitates direct or indirect communication between various elements of the processor circuit 510 and/or the probe 110 (FIG. 1) and/or the host 130 (FIG. 1).

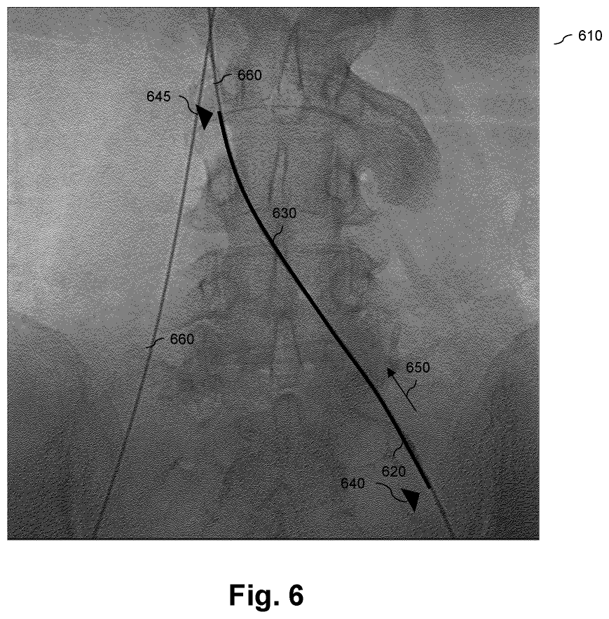

FIG. 6 is a diagrammatic view of an x-ray fluoroscopy image illustrating a pullback procedure, according to aspects of the present disclosure. FIG. 6 depicts an x-ray fluoroscopy image 610 showing an intravascular device 620 and guidewires 660. FIG. 6 additionally depicts an intravascular device path 630, a starting indicator 640, an ending indicator 645, and a directional arrow 650.

During a pullback procedure, one or more guidewires 660 may be positioned within one or more lumens of a patient. Because the guidewire 660 may be constructed of a flexible material, the shape of the guidewire 660 may conform to the shape of the lumen in which the guidewire 660 is positioned. The guidewire 660 may include a flexible elongate member. An intravascular device 620 may be positioned within the lumen and travel through the lumen along a guidewire 660, which is positioned within a guidewire lumen of the intravascular device 620. The intravascular device 620 can be a catheter or a guide catheter. The intravascular device 620 may be an IVUS catheter. The device 620 may be constructed of a flexible material, such that the shape of the device 620 may match the curvature of the lumen in which the device 620 is positioned. The intravascular device 620 may include a flexible elongate member. In the fluoroscopy image 610, a radiopaque portion of the intravascular device 620 is visible. The intravascular device 620 may be substantially similar to the device 102 of the intraluminal ultrasound imaging system 101. A user of the system 100 may position the intravascular device 620 at a starting location shown by the indicator 640. With the intravascular device 620 placed at the starting location, the user may begin acquiring fluoroscopy images with the x-ray imaging system 151. The image 610 may be one of the many x-ray fluoroscopy images obtained during the pullback. In some embodiments, the fluoroscopy image 610 is an x-ray image obtained while no contrast agent is present within the patient anatomy. In such an embodiment, the lumens (e.g., blood vessel) of the patient may be identified primarily by the positioning of the guidewires 660 within the lumens. In other embodiments, the image 610 may be an x-ray image obtained while a contrast agent is present within the patient anatomy. The contrast agent may make vessel lumens visible within the image 610.

One or a plurality of radiopaque portions of the guidewire 660 are visible in the x-ray image(s) 610 obtained without contrast. The radiopaque portions can be one length or a plurality of lengths of the guidewire 660. In some embodiments, the radiopaque portions of the guidewire 660 are one or a plurality of radiopaque markers. The radiopaque markers can be made of a different material that is more radiopaque than the material used to form other parts of the guidewire 660. In some embodiments, all or substantially all of the guidewire 660 can be radiopaque. In some embodiments, all or substantially all of the portion of the guidewire 660 within the patient body can be radiopaque. In some embodiments, all or substantially all of the distal portion of the guidewire 660 (e.g., the portion of the guidewire being imaged by x-ray) can be radiopaque. For example, the guidewire 660 can be sufficiently thick (e.g., a sufficiently large diameter) to provide radiopacity in x-ray images 610. Such embodiments can include clinical applications in the peripheral venous system, which can involve guidewires with a diameter between 0.014″ and 0.038″, including values such as 0.014″, 0.018″, 0.035″, 0.038″, and/or other values both larger and smaller.

While the x-ray imaging system 151 acquires fluoroscopy images, the user of the system 100 may then begin to move device 620 through the patient lumen along the guidewire 660. The user may pull the device in a direction shown by the arrow 650. As the device 620 moves along the guidewire 660 through the lumen, the device 620 shown in newly acquired fluoroscopy images is shown to move in the direction shown by the arrow 650. The user may continue to pull the device 620 along the guidewire 660 until an ending position 645. The path taken by the device 620 during the pullback procedure may be illustrated by the path 630 within FIG. 6.

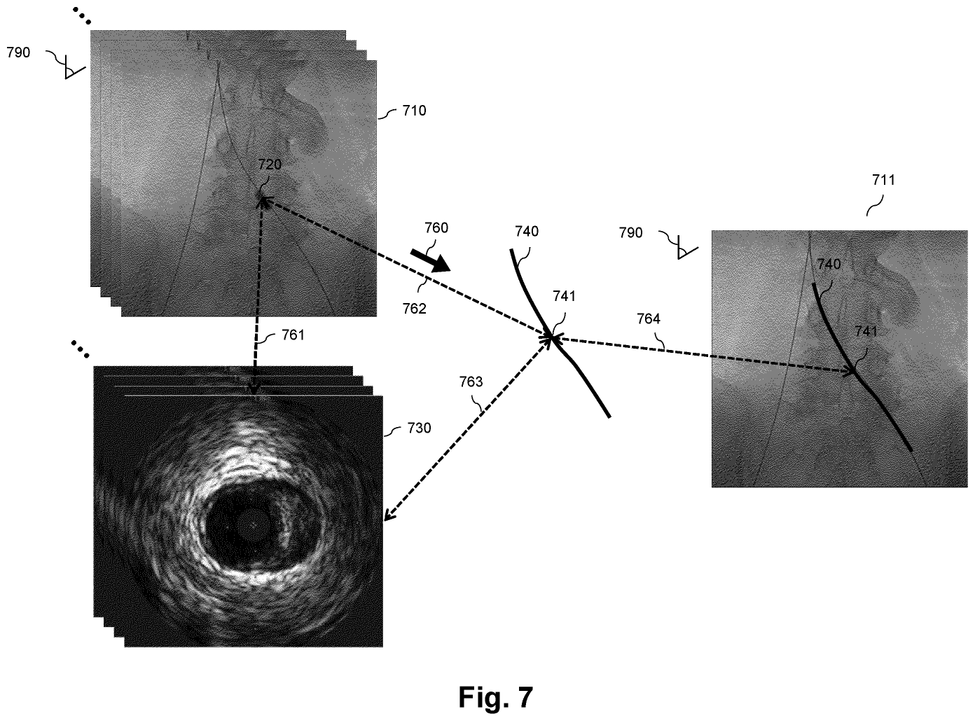

As the device 620 moves from the starting position shown by the indicator 640 to the ending position shown by the indicator 645, it may acquire any suitable intravascular data, such as IVUS images. After the device 620 has moved to the ending position, the user may stop acquiring fluoroscopy images with the x-ray imaging system 151 and may remove the device 620 from the lumen. Because the intravascular data was obtained with the device 620 while fluoroscopy images were simultaneously acquired, the intravascular data may be coregistered to the places along the path 630 at which each datum was collected and displayed in relation to that location along the path 630 and/or a representative fluoroscopy image as will be described with greater detail with reference to FIG. 7.

In some embodiments, the intravascular device 620 may be moved in an opposite direction. For example, the device may be moved from the position of indicator 645 to the position of indicator 640. In other words, the device 620 may move from a distal region to a proximal region (e.g., a pullback) or may move from a proximal region to a distal region (e.g., push forward) during the imaging procedure.

It is noted, that the starting and ending positions may represent target locations during an IVUS imaging procedure. Any indicators, such as indicators 640 and/or 645, identifying these locations may not be visible within an x-ray image displayed to a user during a pullback procedure. For example, during an imaging procedure, the system may identify the starting location of the device 620 on the display, but the ending location of the device 620 is not known because the procedure is still in the process of being completed. However, after an IVUS imaging procedure or pullback procedure is completed, during a review phase of the process, indicators 640 and/or 645 identifying both the starting location and the ending location may be displayed to a user of the system.

FIG. 7 is a diagrammatic view of a relationship between x-ray fluoroscopy images 710, intravascular data 730, and a path 740 defined by the motion of an intravascular device, according to aspects of the present disclosure. FIG. 7 describes a method of coregistering intravascular data 730 including intravascular images with corresponding locations on one or more fluoroscopy images 710 of the same region of a patient's anatomy.

The patient anatomy may be imaged with an x-ray device while a physician performs a pullback with an intravascular device 720, e.g., while the intravascular device 720 moves through a blood vessel of the anatomy. The intravascular device may be substantially similar to the intravascular device 102 described with reference to FIG. 1. The x-ray device used to obtain the fluoroscopy images 710 may be substantially similar to the x-ray device 152 of FIG. 1. In some embodiments, the fluoroscopy images 710 may be obtained while no contrast agent is present within the patient vasculature. Such an embodiment is shown by the fluoroscopy images 710 in FIG. 7. The radiopaque portion of the intravascular device 720 is visible within the fluoroscopy image 710. The fluoroscopy images 710 may correspond to a continuous image stream of fluoroscopy images and may be obtained as the patient anatomy is exposed to a reduced dose of x-radiation. It is noted that the fluoroscopy images 710 may be acquired with the x-ray source 160 and the x-ray detector 170 positioned at any suitable angle in relation to the patient anatomy. This angle is shown by angle 790.

The intravascular device 720 may be any suitable intravascular device. As the intravascular device 720 moves through the patient vasculature, the x-ray imaging system may acquire multiple fluoroscopy images 710 showing the radiopaque portion of the intravascular device 720. In this way, each fluoroscopy image 710 shown in FIG. 7 may depict the intravascular device 720 positioned at a different location such that a processor circuit may track the position of the intravascular device 720 over time.

As the intravascular device 720 is pulled through the patient vasculature, it may acquire intravascular data 730. In an example, the intravascular data 730 shown in FIG. 7 may be IVUS images. However, the intravascular data may be any suitable data, including IVUS images, FFR data, iFR data, OCT images, intravascular photoacoustic (IVPA) images, or any other measurements or metrics relating to blood pressure, blood flow, lumen structure, or other physiological data acquired during a pullback of an intravascular device.

As the physician pulls the intravascular device 720 through the patient vasculature, each intravascular data point 730 acquired by the intravascular device 720 may be associated with a position within the patient anatomy in the fluoroscopy images 710, as indicated by the arrow 761. For example, the first IVUS image 730 shown in FIG. 7 may be associated with the first fluoroscopy image 710. The first IVUS image 730 may be an image acquired by the intravascular device 720 at a position within the vasculature, as depicted in the first fluoroscopy image 710 as shown by the intravascular device 720 within the image 710. Similarly, an additional IVUS image 730 may be associated with an additional fluoroscopy image 710 showing the intravascular device 720 at a new location within the image 710, and so on. The processor circuit may determine the locations of the intravascular device 720 within each acquired x-ray image 710 by any suitable method. For example, the processor circuit may perform various image processing techniques, such as edge identification of the radiopaque marker, pixel-by-pixel analysis to determine transition between light pixels and dark pixels, filtering, or any other suitable techniques to determine the location of the imaging device 720. In some embodiments, the processor circuit may use various artificial intelligence methods including deep learning techniques such as neural networks or any other suitable techniques to identify the locations of the imaging device 720 within the x-ray images 710.

Any suitable number of IVUS images or other intravascular data points 730 may be acquired during an intravascular device pullback and any suitable number of fluoroscopy images 710 may be obtained. In some embodiments, there may be a one-to-one ratio of fluoroscopy images 710 and intravascular data 730. In other embodiments, there may be differing numbers of fluoroscopy images 710 and/or intravascular data 730. The process of co-registering the intravascular data 730 with one or more x-ray images may include some features similar to those described in U.S. Pat. No. 7,930,014, titled, “VASCULAR IMAGE CO-REGISTRATION,” and filed Jan. 11, 2006, which is hereby incorporated by reference in its entirety. The co-registration process may also include some features similar to those described in U.S. Pat. Nos. 8,290,228, 8,463,007, 8,670,603, 8,693,756, 8,781,193, 8,855,744, and 10,076,301, all of which are also hereby incorporated by reference in their entirety.

The system 100 may additionally generate a fluoroscopy-based 2D pathway 740 defined by the positions of the intravascular device 720 within the x-ray fluoroscopy images 710. The different positions of the intravascular device 720 during pullback, as shown in the fluoroscopy images 710, may define a two-dimensional pathway 740, as shown by the arrow 760. The fluoroscopy-based 2D pathway 740 reflects the path of one or more radiopaque portions of the intravascular device 720 as it moved through the patient vasculature as observed from the angle 790 by the x-ray imaging device 152. The fluoroscopy-based 2D pathway 740 defines the path as measured by the x-ray device which acquired the fluoroscopy images 710, and therefore shows the path from the same angle 790 at which the fluoroscopy images were acquired. Stated differently, the 2D pathway 740 describes the projection of the 3D path followed by the device onto the imaging plane at the imaging angle 790. In some embodiments, the pathway 740 may be determined by an average of the detected locations of the intravascular device 720 in the fluoroscopy images 710. For example, the pathway 740 may not coincide exactly with the guidewire in any fluoroscopy image 710 selected for presentation.

As shown by the arrow 762, because the two-dimensional path 740 is generated based on the fluoroscopy images 710, each position along the two-dimensional path 740 may be associated with one or more fluoroscopy images 710. As an example, at a location 741 along the path 740, the first fluoroscopy image 710 may depict the intravascular device 720 at that same position 741. In addition, because a correspondence was also established between the fluoroscopy images 710 and the intravascular data 730 as shown by the arrow 761, intravascular data 730, such as the first IVUS image shown, may also be associated with the location 741 along the path 740 as shown by the arrow 763.

Finally, the path 740 generated based on the locations of the intravascular device 720 within the fluoroscopy images 710 may be overlaid onto any suitable fluoroscopy image 711 (e.g., one of the fluoroscopic images 710 in the fluoroscopic image stream). In this way, any location along the path 740 displayed on the fluoroscopy image 711 may be associated with IVUS data such as an IVUS image 730, as shown by the arrow 764. For example, IVUS image 730 shown in FIG. 7 may be acquired simultaneously with the fluoroscopy image 710 shown and the two may be associated with each other as shown by the arrow 761. The fluoroscopy image 710 may then indicate the location of the intravascular device 720 along the path 740, as shown by the arrow 762, thus associating the IVUS image 730 with the location 741 along the path 740 as shown by the arrow 763. Finally, the IVUS image 730 may be associated with the location within the fluoroscopy image 710 at which it was acquired by overlaying the path 740 with associated data on the fluoroscopy image 711. The pathway 740 itself may or may not be displayed on the image 711.

In the illustrated embodiment of FIG. 7, the co-registered IVUS images are associated with one of the fluoroscopic images obtained without contrast such that that the position at which the IVUS images are obtained is known relative to locations along the guidewire. In other embodiments, the co-registered IVUS images are associated with an x-ray image obtained with contrast (in which the vessel is visible) such that that the position at which the IVUS images are obtained is known relative to locations along the vessel.

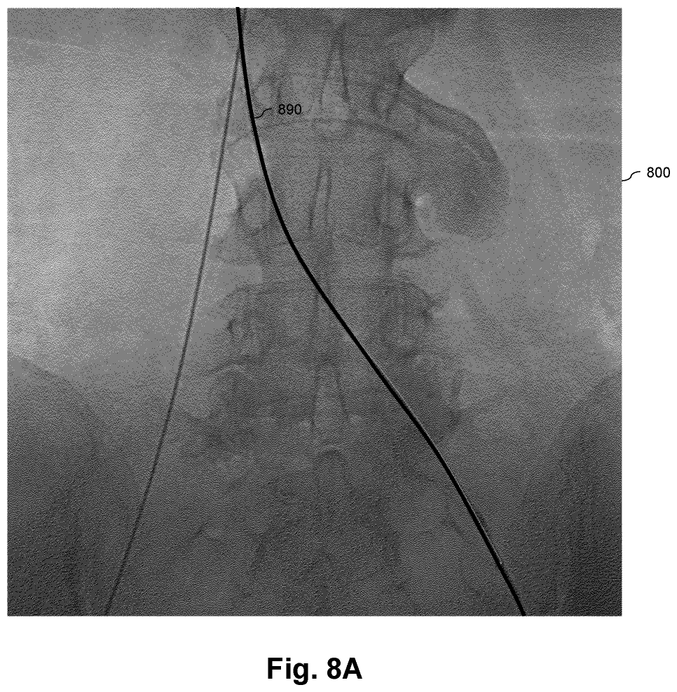

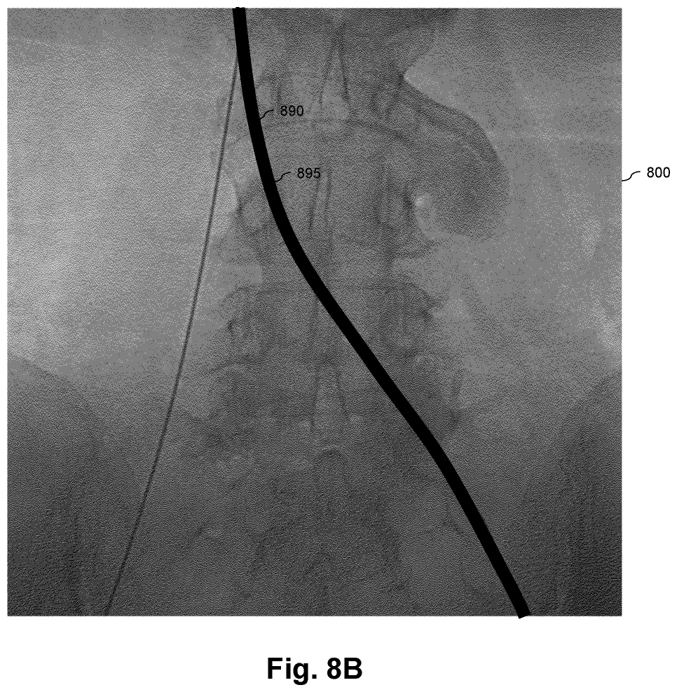

FIG. 8A is a diagrammatic view of an x-ray image 800 identifying a guidewire 890, according to aspects of the present disclosure. The x-ray image 800 shown in FIG. 8A may be substantially similar to the x-ray images 610, 710, and/or 711 previously described. For example, the x-ray image 800 may be an x-ray image acquired by the x-ray imaging system 151 (FIG. 1). The x-ray image 800 may be displayed to a user of the system 100 via the display 132. The x-ray image 800 may alternatively not be displayed to a user of the system 100 via the display 132. The guidewire 890 is visible in the image 800. The guidewire 890 can be a continuous flexible elongate member that is positioned within a blood vessel inside the patient body. For clarity, the guidewire is depicted as a solid line in FIG. 8A. The guidewire 890 may be substantially similar to the guidewire 660 described previously (FIG. 6). For example, the guidewire 890 may be constructed of a radiopaque material. As a result, the guidewire 890 may appear within an x-ray fluoroscopy image in which no radiopaque contrast agent has been introduced to the patient vasculature.

FIG. 8B is a diagrammatic view of the x-ray image 800 of FIG. 8A that identifies a lumen 895, such as a blood vessel, in which the guidewire 890 is positioned. The lumen 895 may be represented in FIG. 8B by a solid line of greater width than the solid line illustrating the guidewire 890 discussed with reference to FIG. 8A. The lumen 895 may be any suitable lumen within the patient anatomy. The lumen 895 may be a blood vessel. In an application in which no contrast agent is introduced to the patient anatomy, the lumen 895 may not be ordinarily visible within the x-ray image 800. For example, the patient anatomy shown in the x-ray image 800 may contain multiple blood vessels throughout the region shown. However, without a contrast agent, these vessels may not appear within the image 800. The lumen 895 is artificially highlighted in FIG. 8B by the solid white line for the purpose of explaining the present disclosure. In particular, the guidewire 890 has the same shape of the lumen 895 in which it is positioned.

Prior to an imaging procedure, the guidewire 890 may be positioned within the lumen 895 as shown in FIG. 8B. The imaging device 620 (FIG. 6) may then be positioned so as to move along the guidewire 890. The imaging device 620 may be substantially similar to the device 102 (FIG. 4). For example, the device 620 may include a lumen similar to the lumen 236 (FIG. 4), through which the guidewire 890 is received. As the device 620 moves along the guidewire 890, it moves within the vessel 895 imaging, or acquiring other intravascular data relating to, the vessel 895. Because the guidewire 890 is positioned within the lumen 895, the device 620 moves within the vessel 895 at all times. The guidewire 890 can remain stationary within the vessel 895 as the imaging device 620 moves along the guidewire 890 to obtain IVUS images of the vessel.

Because the guidewire 890 is positioned within the lumen 895, the guidewire 890 indicates the location of the lumen 895 within the image 800. For example, the guidewire 890 would be in the same location, of the same shape, profile, or orientation as the lumen 895 in which it is placed. Because the guidewire 890 is constructed of radiopaque material, it appears within an x-ray image 800 without contrast being introduced to the patient anatomy. The processor circuit of the system 100 may then indirectly identify the vessel 895 within an image 800 without introducing a contrast agent to the vasculature because the processor circuit of the system 100 can directly identify the guidewire 890. Thus, the system 100 determines the location of the vessel 895 within the x-ray image by identifying the location of the guidewire 890. The system 100 may employ various image processing techniques to identify the guidewire 890 within an image 800. For example, the system 100 may use image processing techniques such as edge detection, image editing or restoration, linear filtering or other filtering methods, image padding, or any other suitable image processing techniques. For example, the system 100 can use a pixel-by-pixel analysis to identify longitudinally adjacent dark pixels along the length of guidewire. Each of the dark pixels can be laterally adjacent to light pixels representative of the vessel 895. In some embodiments, the system 100 may use deep learning techniques to identify the location of the guidewire 890 within an image 800.

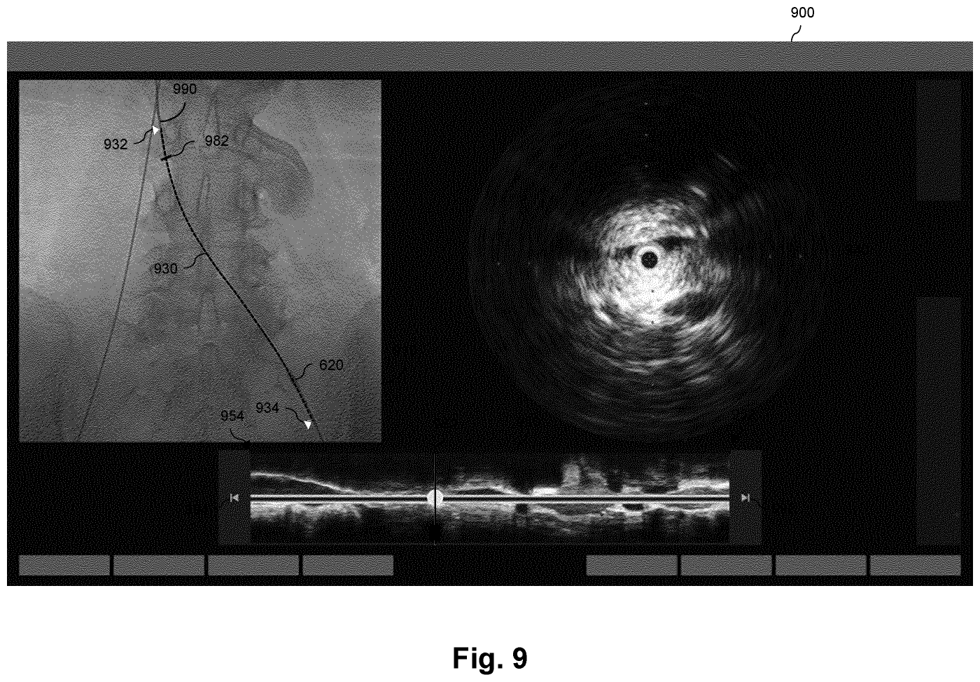

FIG. 9 is a diagrammatic view of a graphical user interface 900 displaying an intravascular image 940 coregistered to an x-ray image 910, according to aspects of the present disclosure. The graphical user interface 900 may include an x-ray image 910, an IVUS image 940, and a longitudinal view 950 of the vessel. The longitudinal view 950 can be an ILD, such as an in-line digital or image longitudinal display.

The x-ray image 910 may be displayed to a user of the system 100 within the graphical user interface 900. The x-ray image 910 may be substantially similar to the image 800 discussed with reference to FIG. 8A and FIG. 8B. The x-ray image 910 may be acquired with the x-ray imaging system 151 and may be received by the processor 134 of the system 100 (FIG. 1). Similar to the image 800, the x-ray image 900 may be an x-ray image acquired during an imaging procedure in which no contrast agent is added to the patient vasculature. The image 910 may be one of many x-ray images acquired in a continuous image stream. The x-ray image 910 may be an x-ray fluoroscopy image. In other embodiments, different types of x-ray images may be used. The x-ray image 910 provides the user with a view of a region of the patient anatomy through which the intravascular device 620 moved during an imaging procedure.

In some embodiments, the x-ray image 910 shown in the interface 900 is one of the x-ray images obtained by the x-ray imaging system 151 during the IVUS pullback procedure. In other embodiments, however, the x-ray image 910 may not be one of the x-ray images obtained during the pullback procedure. For example, the x-ray image 910 may be any suitable image acquired of the same region of the patient with a guidewire positioned within the same vessel imaged. In such an embodiment, the x-ray image 910 may be acquired from a similar angle as the x-ray images acquired during the procedure such that the shape, placement, orientation, and general appearance of the guidewire within the image 910 is similar to the pathway defined by the movement of the intravascular device 620 during the imaging procedure.

The system 100 may receive from the x-ray imaging system 151 a plurality of x-ray images. Some of these images may have been acquired as the pullback procedure was performed. In other words, some of the received x-ray images may have been received while the intravascular device 620 was acquiring IVUS images. However, some x-ray images received may not have been acquired during the pullback procedure. Rather, some may have been acquired before or after the pullback procedure.

In some embodiments, the x-ray image 910 may include a depiction of a radiopaque portion of the intravascular device 620, as shown in FIG. 9. Because the intravascular device 620 is constructed of radiopaque material, it may be visible in the image 910 acquired without contrast agent. For example, the portion of the intravascular device 620 that is visible in the x-ray image 910 can be the imaging assembly (e.g., transducer assembly) and/or radiopaque markers.

The image 910 may additionally depict one or more guidewires 990. As discussed with reference to FIG. 8A and FIG. 8B, the guidewire 990 may be constructed of radiopaque material such that it appears within the x-ray image 910. Because the guidewire 990 is positioned within the lumen to be imaged, it indicates the location of the vessel within the image 910. Any suitable number of guidewires 990 may be displayed within an x-ray image. For example, two guidewires 990 are shown within the image 910. Additional guidewires 990 may also be present.

The graphical user interface 900 may correspond to a display presented to the user of the system 100 during or after a pullback procedure. A pullback procedure may include an imaging procedure in which the intravascular device 620 is moved through the patient anatomy along the guidewire 990 within a lumen while the x-ray imaging system 151 simultaneously acquires fluoroscopy images of the same region of the patient anatomy without contrast agent inside the vessel. The marker 934 within the fluoroscopy image 910 may indicate a starting position of the intravascular device 620 at the beginning of the pullback procedure. For example, the marker 934 may identify the location along the guidewire 990 at which the first IVUS image was obtained during the pullback imaging procedure. Similarly, the marker 932 may indicate an ending position of the intravascular device 620 at the end of the pullback procedure. For example, the marker 932 may identify the location along the guidewire 990 at which the final IVUS image was obtained during the pullback imaging procedure. In this example, the pullback procedure may include moving the intravascular device 620 through the vessel from a location represented by the marker 934 to the location represented by the marker 932 while the device 620 acquires intravascular data. The system 100 can, e.g., automatically track movement of the radiopaque portion of the device 620 in the plurality of x-ray images acquired as the device 620 moves within the vessel. The intravascular device 620 may move from a distal location within a vessel to a proximal location, as described, or it may move in the opposite direction. For example, the marker 932 may indicate a starting location of the device 620 and the marker 934 may indicate an ending location.

A pathway 930 is also shown overlaid over the x-ray image 910. The pathway 930 may be similar to the pathway 630 described with reference to FIG. 6 or the pathway 740 described with reference to FIG. 7. For example, the pathway 930 may be determined and generated by the system 100 based on the locations of the radiopaque portion of the intravascular device 620 within the x-ray images acquired by the x-ray imaging system 151. The location of the device 620 may be determined by the system 100 using any above-mentioned image processing or deep learning techniques for each acquired x-ray image. These locations may together define the shape of the pathway 930 overlaid over the image 910. In this way, the length of the pathway 930 shown on the image 910 may correspond to the length along the vessel which was imaged by the intravascular device 620. Because the imaging device 620 moved along the guidewire 990, this pathway 930 is similar in shape to the corresponding section of the guidewire 990 representative of the imaged vessel as shown in FIG. 9. In some embodiments, the pathway 930 may not be shown. Indeed, any of the pathway 930 and the indicators 932 and 934 may not be displayed to the user within the graphical user interface 900. In addition, any of the pathway 930 and indicators 932 and 934 may appear differently than they appear in FIG. 9.

In some embodiments, the processor circuit of the system 100 may use some or a subset of the plurality of x-ray images received by the system during the pullback procedure to determine the path 930 of movement of the intravascular device 620 and/or co-registration of intravascular data to corresponding locations within the x-ray image 910. In some embodiments, all of the plurality of x-ray images received are used by the processor circuit to determine the path 930 and complete the coregistration. However, some of the x-ray images acquired may not depict the radiopaque portion of the device 620 or may have been acquired at a time when the device 620 was not acquiring IVUS images.

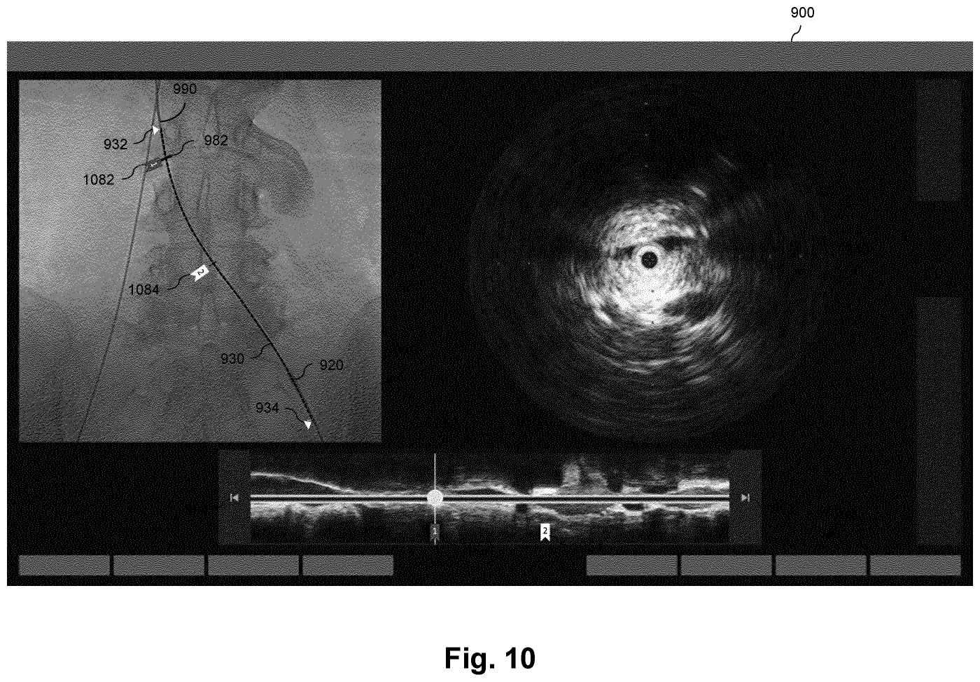

The process of coregistering intravascular data to locations within the x-ray image 910 along the guidewire 990 may include first co-registering the data to the pathway 930. For example, as explained with reference to FIG. 7, the plurality of IVUS images acquired by the device 620 may be coregistered to locations along the pathway 740. With reference to FIG. 9, using similar techniques, the acquired IVUS images may be coregistered to the pathway 930 shown in the interface 900. The pathway 930 is overlaid over the x-ray image 910 at the corresponding location as the guidewire 990. Because the pathway 930 is of the same shape as the guidewire 990 such that the pathway 930 aligns with the guidewire 990 throughout the length of imaged region of the vessel, the intravascular data that was coregistered to the pathway 930 may be additionally coregistered to the guidewire 990. As a result, the pathway 930 may be displayed or may not be. The indicator 982 and/or bookmarks, discussed with reference to FIG. 10, may be displayed with relation to the guidewire 990 without the pathway 930 being displayed.

The image 910 may include an indicator 982. The graphical user interface 900 additionally includes the IVUS image 940 displayed adjacent to the x-ray image 910. The indicator 982 may identify to the user of the system 100 the location along the guidewire 990 or pathway 930 at which the IVUS image 940 was obtained. Because the guidewire 990 is positioned within the imaged vessel, as the indicator 982 is displayed along the guidewire 990, it also identifies to the user the location along the imaged vessel at which the IVUS image 940 was acquired. Thus, even though the vessel cannot be directly visualized because the x-ray images were obtained without contrast, registration of the IVUS image to a corresponding location along the vessel is still possible because the guidewire 990 acts as the vessel. The indicator 982 is disposed along the guidewire 990, not the vessel, in the x-ray image 910 at a corresponding position of the IVUS image 940.

The indicator 982 may be of any suitable appearance and may be positioned at any suitable location relative to the guidewire 990. For example, the indicator 982 may be a single solid line, as shown. Alternatively, the indicator 982 may be of any suitable shape, profile, color, pattern, weight, or other appearance. The indicator 982 may be positioned overlaid on the guidewire 990 or the pathway 930 or may be positioned elsewhere. For example, the indicator 982 may be positioned adjacent to the guidewire 990 or the pathway 930. The indicator 982 may be positioned along an axis perpendicular to the guidewire 990 or the pathway 930 and spaced from the guidewire 990 or pathway 930 or positioned in any other location so as to indicate the location along the guidewire 990 or pathway 930 the location at which the IVUS image 940 was acquired. All or part of the indicator 982 may be positioned over the guidewire 990, adjacent to the guidewire 990, proximate to the guidewire 990, spaced from the guidewire 990, or combinations thereof. The indicator 982 may also be referred to as a marker, marking, identifier, scrubber, pointer, or any other suitable term.

Earlier coregistration systems required a roadmap x-ray image of a vessel that is obtained with contrast. The locations of coregistered IVUS images were displayed in the roadmap image relative to the contrast-filled vessel in these earlier systems. This required more time for the procedure because the contrast had to be introduced into the vessel and the x-ray image with contrast had to be taken. The present disclosure advantageously avoids the prolongation of the IVUS imaging procedure caused with the contrast and the potential patient discomfort associated with this delay and/or the contrast itself. In addition, some patients may be more sensitive to contrast due to various conditions. Such conditions may include, among others, impaired kidney function. Avoiding radiopaque contrast in such situations is clinically advantageous because it avoids risk of harm to the patient. In particular, the roadmap x-ray image 910 in the present disclosure does not have to be obtained with contrast or have a contrast-filled vessel. Rather, the roadmap x-ray image 910 can be obtained without contrast because the guidewire 990 that is positioned within the vessel is visible in the roadmap x-ray image. The marker or marking 982 is displayed relative to the guidewire 990, not the vessel.

The graphical user interface 900 additionally depicts an ILD 950. The IVUS images acquired with the device 620, may be used to create an ILD 950, shown adjacent to the IVUS image 940. In that regard, IVUS image 940 is a tomographic or radial cross-sectional view of the blood vessel. The ILD 950 provides a longitudinal cross-sectional view of the blood vessel. The ILD 950 can be a stack of the IVUS images acquired at various positions along the vessel, such that the longitudinal view of the ILD 950 is perpendicular to the radial cross-sectional view of the IVUS image 940. In such an embodiment, the ILD 950 may show the length of the vessel, whereas an individual IVUS image 940 is a single radial cross-sectional image at a given location along the length. In another embodiment, the ILD 950 may be a stack of the IVUS images acquired overtime during the imaging procedure and the length of the ILD 950 may represent time or duration of the imaging procedure. The ILD 950 may be generated and displayed in real time or near real time during the pullback procedure. As each additional IVUS image 940 is acquired by the device 620, it may be added to the ILD 950. For example, at a point in time during the pullback procedure, the ILD 950 shown in FIG. 9 may be partially complete. In some embodiments, the processor circuit may generate an illustration of a longitudinal view of the vessel being imaged based on the received IVUS images. For example, rather than displaying actual vessel image data as the ILD 950 does, the illustration may be a stylized version of the vessel, with e.g., continuous lines showing the lumen border and vessel border.

The ILD 950 may include an indicator 962. The indicator 962 may indicate to the user the location along the ILD 950 at which the IVUS image 940 was obtained. The indicator 962 may therefore correspond to the indicator 982. In some embodiments, the processor circuit may move the indicator 982 from one location along the guidewire 990 to another in response to a user input designating the new location. As the indicator 982 is moved to a different location, the indicator 962 may be moved to the corresponding location along the ILD 950 and the IVUS image acquired at the location may be displayed. In this way, the location at which the IVUS image shown in the graphical user interface 900 may be shown within the x-ray image 910 by the indicator 982 and within the ILD 950 by the indicator 962. Similarly, if the processor circuit moves the indicator 962 within the ILD 950 in response to a user input, the indicator 982 may move to the corresponding location along the guidewire 990 within the image 910 and the IVUS image acquired at that new location may be displayed.

The processor circuit may move the indicator 982 by any suitable method or in response to any type of user input. For example, the user may use a mouse to click on a location along the guidewire 990, may touch a location along the guidewire 990 using a touchscreen device, or may indicate the new location by any other way. In some embodiments, the user may select and drag the indicator 982 to a different location along the guidewire 990. Similarly, the user may move the indicator 962 to different locations along the ILD 950 by any of these methods as well. The system 100 may also display different regions of the ILD 950 in response to a user input selecting either of the arrow 992 or the arrow 994. In some embodiments, the system may also display a different IVUS image than the image 940 in response to a user selection of the arrows 992 or 994 and move the indicator 982 to a different location along the guidewire 990. For example, selection of the arrows 992 or 994 can provide backwards and forwards frame by frame scrolling for greater navigation accuracy.

The indicator 962 displayed on the ILD 950 may be of any suitable appearance or positioned at any suitable location. For example, the indicator 962 may include a line extending perpendicularly across the ILD 950 as shown. The indicator 962 may include any suitable shape, such as a circle positioned at a central point along the line as shown. In other embodiments, the indicator may be of any pattern, weight, color, shape, profile, or any other appearance. The indicator 962 may be positioned over the ILD 950 as shown, or may be positioned next to the ILD 950 or positioned at any other suitable location so as to indicate the location along the ILD 950 at which the corresponding IVUS image shown was acquired.

The indicator 982 and/or the indicator 962 may alternatively be referred to by any suitable term, including but not limited to a scrubber, marker, marking, pointer, or by any other suitable term.

FIG. 10 is a diagrammatic view of a graphical user interface displaying an intravascular image 940 coregistered to an x-ray image 910, according to aspects of the present disclosure. The x-ray image 910 and the ILD 950 shown in the graphical user interface 900 of FIG. 10 may include one or more annotations including bookmarks 1082, 1084, 1052, and 1054.

During or after an imaging procedure, the processor circuit of the system 100 may create annotations related to the acquired data in response to a user input. The annotations created may include any information, including text, symbols, images, or any other content. The annotations may identify regions of interest along the imaged vessel in the x-ray image, or in any acquired IVUS image. Annotations may mark IVUS images individually. For example, one of the IVUS images may display a region along the imaged vessel with greatest constriction, a minimum lumen area, a minimum lumen diameter, a proximal landing zone (e.g., healthy tissue proximal of the blood flow constriction) for the proximal stent edge, a distal landing zone (e.g., healthy tissue distal of the blood flow constriction) for the distal stent edge, locations where two vessels join together or split apart, etc. The user may wish to identify an image to be easily located again. Annotations may identify or highlight sections of an IVUS image, an x-ray image, or the ILD 950.

In some embodiments, annotations may include bookmarks. In an embodiment in which a feature of interest is observed by the user of the system 100 within an IVUS image, the processor circuit may create a bookmark corresponding to the image in response to a user input identifying a location or image. For example, in FIG. 10, the user may wish to identify the IVUS image 940 shown with a bookmark. The image 940 may identify an occlusion within the vessel or any other features of interest. The user input received by the processor circuit may be of any suitable type, including those previously described. For example, the user input may be a selection of the buttons 1050 within the interface 900 directing the system 100 to create a bookmark. After the IVUS image 940 is identified, a bookmark 1052 may be positioned along the ILD 950. This bookmark 1052 may identify where along the ILD 950 the identified image 940 is located within the vessel as illustrated by the ILD 950. As the indicator 952 and the corresponding indicator 982 are moved to different locations and different IVUS images are displayed in the place of image 940, the bookmark 1052 may remain at the same location along the ILD 950. A user of the system 100 may then select the bookmark 1052 to quickly move the indicator 952 to the same location as the bookmark 1052 along the ILD 950 as shown and cause the previously identified IVUS image 940 to be displayed within the interface 900.

The processor circuit may automatically generate an additional bookmark 1082 to be placed within the x-ray image 910 corresponding to the bookmark 1052. Just as the indicator 982 identifies the location along the guidewire 990 corresponding to the location shown by the indicator 952 along the ILD 950, the bookmark 1082 may identify the location along the guidewire 990 at which the identified and bookmarked image 940 was acquired. The processor circuit can automatically provide the bookmark 1082 in the x-ray image in response to the IVUS image 940 being bookmarked (e.g., the processor circuit generating the bookmark 1082 in response to the user input to create the bookmark 1082). In that regard, the bookmark 1082 is displayed relative to the guidewire 990 in the x-ray image 910, not the vessel because the vessel cannot be visualized without contrast agent. Similarly, a user may select the bookmark 1082 along the guidewire, not along the vessel. Upon this selection, the processor circuit may cause the indicator 982 to move to the same location along the guidewire 990 as the bookmark 1082. The indicator 952 may also be moved to the same location along the ILD 950 as the corresponding bookmark 1052 and the IVUS image 940 may be displayed.

The bookmark 1082 may be a graphical representation overlaid on the x-ray image 910. The processor circuit may generate and display the bookmark 1082 in response to a user input designating the location along the guidewire 990. The bookmark 1082 may be of any suitable appearance and be positioned at any suitable location with regard to the x-ray image 910. For example, the bookmark 1082 may be of any suitable shape, color, pattern, or size, and may include any alphanumeric text. In one embodiment, the bookmark 1082 may include a flag with a numeral. The numeral may correspond to the order in which the bookmark 1082 was created by the processor circuit in relation to other generated bookmarks. The bookmark 1082 may include any other suitable text describing the bookmark 1082, the location at which the bookmark 1082 is positioned, or any other features of the patient anatomy. The bookmark 1082 may be positioned to the side of the guidewire 990 as shown. The bookmark 1082 may be positioned at some location spaced from the guidewire 990. The bookmark 1082 may also be positioned adjacent to the indicator 982. In some embodiments, the bookmark 1082 may overlap or be positioned directly next to the indicator 982. The bookmark 1082 may also be at any other suitable location, including proximate to, adjacent to, or overlapping the guidewire 990.

In some embodiments, the processor circuit of the system 100 may identify IVUS images of interest automatically. The system 100 may identify an IVUS image acquired along the imaged vessel showing an occlusion, a lesion, or any other relevant feature. When the system 100 identifies an IVUS image of interest, it may similarly automatically create a bookmark 1054 along the ILD 950. The bookmark 1054 may be similar to the bookmark 1052 in that it may identify where along the ILD 950 the automatically identified IVUS image is located within the vessel on the ILD 950. As the user navigates to different IVUS images, the bookmark 1054 may remain at the same location. The user may then select the bookmark 1054 to quickly move the indicator 952 to the same location along the ILD 950 and cause the automatically identified IVUS image to be displayed. In some aspects, the system 100 may automatically identify and create bookmarks relating to IVUS images using some features similar to those described in U.S. Publication No. 2020/0129148, titled “Intraluminal Ultrasound Imaging with Automatic and Assisted Labels And Bookmarks,” and U.S. Provisional Application No. 62/969,857, filed Feb. 4, 2020, and titled “Automatic Intraluminal Imaging-Based Target and Reference Image Frame Detection and Associated Devices, Systems, and Methods,” each of which is hereby incorporated by reference in its entirety.

A similar relationship may exist between the bookmarks 1054 and 1084 as the bookmarks 1052 and 1082. The bookmark 1084 may be automatically placed within the x-ray image 910 corresponding to the bookmark 1054 and may identify the location along the guidewire 990 at which the automatically identified IVUS image was acquired. The processor circuit can automatically provide the bookmark 1084 in the x-ray image in response to the corresponding IVUS image being automatically bookmarked (e.g., the processor circuit generating the bookmark 1082 in response to automatically determining an IVUS image frame to bookmark). Similar to the bookmark 1082, the bookmark 1084 may be of any suitable appearance and may be positioned at any suitable location with regard to the x-ray image 910, the guidewire 990, or the indicator 982. The bookmark 1084 may incorporate any of the features described with reference to the bookmark 1082. Because of the co-registration, the IVUS bookmarks (generated manually or automatically) are automatically transferred to the x-ray display. This saves time and is more accurate for the user, who would otherwise need to manually place them on the x-ray as well. For example, because of co-registration, the corresponding location of an IVUS bookmark on the x-ray image is also automatically identified. Hence, in some embodiments, a fully automated process can generate bookmarks on IVUS and identify and mark the corresponding locations on the X-ray image. In this way, the identification of regions of interest within the x-ray image 910 and/or the IVUS image 940 and the generation of corresponding bookmarks 1054 and 1084 may represent a fully automated process and may not require any user input.

The bookmarks 1052, 1082, 1054, and 1084 may be of any suitable appearance. For example, they may include various shapes, patterns, colors, text, or numbers. In some embodiments, bookmarks that are manually created may be of one appearance and automatically created bookmarks may be of another. For example, bookmarks that are created manually by the user may be of on color while automatically created bookmarks are of a different color. In some embodiments, the appearance of the bookmarks may be determined by the user and adjusted in real time to reflect various features or attributes of the identified IVUS images and their corresponding locations along the guidewire 990 and/or the ILD 950. The bookmarks 1052, 1082, 1054, and 1084 may also be referred to as indicators, markings, markers, or any other suitable term.

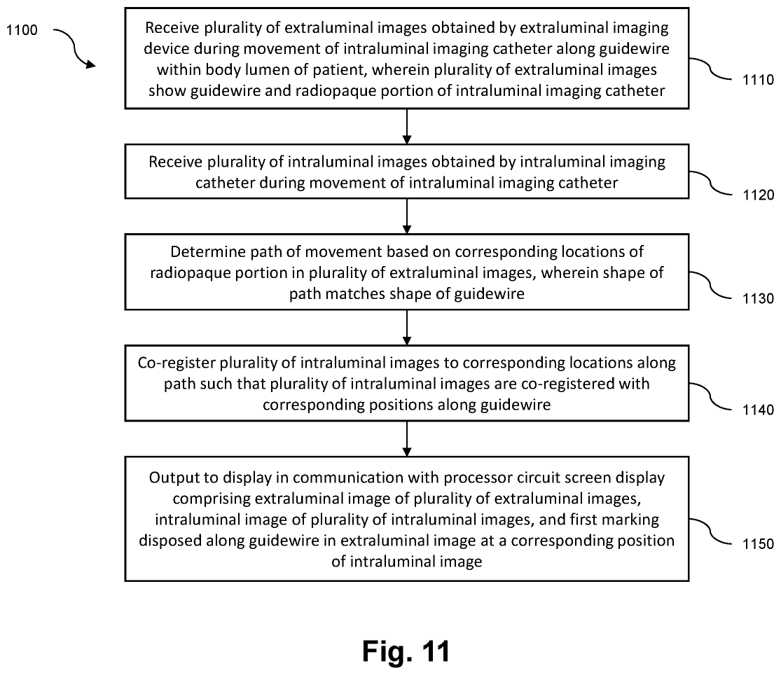

FIG. 11 is a flow diagram for a method 1100 for co-registering intravascular data and/or annotations to locations along a guidewire within an x-ray image obtained without contrast, according to aspects of the present disclosure. As illustrated, the method 1100 includes a number of enumerated steps, but embodiments of the method 1100 may include additional steps before, after, or in between the enumerated steps. In some embodiments, one or more of the enumerated steps may be omitted, performed in a different order, or performed concurrently. The steps of the method 1100 can be carried out by any suitable component within the system 100 and all steps need not be carried out by the same component. In some embodiments, one or more steps of the methods 1100 can be performed by, or at the direction of, a processor circuit of the system 100 (e.g., the processor circuit 510 of FIG. 5), including, e.g., the processor 560 or any other component.

At step 1110, the method 1100 includes receiving a plurality of extraluminal images obtained by the extraluminal imaging device during movement of the intraluminal imaging catheter along the guidewire within the body lumen of a patient. In some embodiments, the plurality of extraluminal images show the guidewire and the radiopaque portion of the intraluminal imaging catheter. For example, step 1110 can include receiving a plurality of x-ray images obtained by the x-ray imaging device during movement of the IVUS imaging catheter along a guidewire within a blood vessel of a patient. In some embodiments, the plurality of x-ray images are obtained without a contrast agent within the blood vessel, and wherein the plurality of x-ray images show the guidewire and a radiopaque portion of the IVUS imaging catheter.

At step 1120, the method 1100 includes receiving a plurality of intraluminal images obtained by the intraluminal imaging catheter during movement of the intraluminal imaging catheter. For examples, step 1120 can include receiving a plurality of IVUS images obtained by the IVUS imaging catheter during the movement of the IVUS imaging catheter.

At step 1130, the method 1100 includes determining a path of movement based on corresponding locations of the radiopaque portion in the plurality of extraluminal images. In some embodiments, the shape of the path matches the shape of the guidewire. For example, step 1130 can include determining a path of the movement based on corresponding locations of the radiopaque portion in the plurality of x-ray images. In some embodiments, a shape of the path matches a shape of the guidewire.

At step 1140, the method 1100 includes co-registering the plurality of intraluminal images to corresponding locations along the path such that the plurality of intraluminal images are co-registered with corresponding positions along the guidewire. For example, the step 1140 can include co-registering the plurality of IVUS images to corresponding locations along the path such that the plurality of IVUS images are co-registered with corresponding positions along the guidewire.

At step 1150, the method 1100 includes outputting to a display in communication with the processor circuit screen a display comprising an extraluminal image of the plurality of extraluminal images, an intraluminal image of plurality of intraluminal images, and a first fmarking disposed along the guidewire in the extraluminal image at a corresponding position of the intraluminal image. For example, step 1150 can include outputting, to a display in communication with the processor circuit, a screen display comprising an x-ray image of the plurality of x-ray images, an IVUS image of the plurality of IVUS images, and a first marking along the guidewire in the x-ray image at a corresponding position of the IVUS image.

Persons skilled in the art will recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A system, comprising:
a processor circuit configured for communication with an extraluminal imaging device and an intraluminal imaging catheter, wherein the processor circuit is configured to:
receive a plurality of extraluminal images obtained by the extraluminal imaging device during movement of an intraluminal imaging catheter along a guidewire within a body lumen of a patient, wherein the plurality of extraluminal images are obtained without a contrast agent within the body lumen, wherein the plurality of extraluminal images show the guidewire and a radiopaque portion of the intraluminal imaging catheter;
receive a plurality of intraluminal images obtained by the intraluminal imaging catheter during the movement of the intraluminal imaging catheter;
co-register the plurality of intraluminal images to corresponding positions along the guidewire based on the plurality of extraluminal images; and
output, to a display in communication with the processor circuit, a screen display comprising:
an extraluminal image of the plurality of extraluminal images;
an intraluminal image of the plurality of intraluminal images; and
a first marking disposed along the guidewire in the extraluminal image at a corresponding position of the intraluminal image.

2. The system of claim 1, wherein the processor circuit is configured to:
receive a user input selecting a different position along the guidewire in the extraluminal image; and
modify the screen display to:
output the intraluminal image of the plurality of intraluminal images corresponding to the different position; and
move the first marking to the different position along the guidewire in the extraluminal image.

3. The system of claim 1,
wherein the screen display comprises:
a longitudinal view of the body lumen based on the plurality of intraluminal images; and
a second marking at a site in the longitudinal view associated with the corresponding position of the intraluminal image;
wherein the processor circuit is configured to:
receive a user input selecting a different site along the longitudinal view; and
modify the screen display to:
move the first marking to a different position along the guidewire in the extraluminal imaging corresponding to the different site;
output the intraluminal image of the plurality of intraluminal images corresponding to the different position along the guidewire in the extraluminal image; and
move the second marking to the different site along the longitudinal view.

4. The system of claim 1, wherein at least a portion of the first marking is disposed over the guidewire in the extraluminal image.

5. The system of claim 1, wherein at least a portion of the first marking is spaced from the guidewire in the extraluminal image.

6. The system of claim 1,
wherein the processor circuit is configured to determine a path of the movement based on corresponding locations of the radiopaque portion in the plurality of x-ray images, wherein a shape of the path matches a shape of the guidewire, and
wherein the processor circuit is configured to co-register the plurality of intraluminal images to corresponding locations along the path, and
wherein the processor circuit is configured to co-register the plurality of intraluminal images to the corresponding positions along the guidewire based on co-registering the plurality of IVUS images to the corresponding locations along the path.

7. The system of claim 6, wherein the screen display comprises:
a graphical representation of the path in the extraluminal image.

8. The system of claim 7, wherein the first marking is disposed along the graphical representation of the path in the extraluminal image.

9. The system of claim 7, wherein the screen display comprises:
a second marking in the extraluminal image representative of a beginning of the path; and
a third marking in the extraluminal image representative of the end of the path.

10. The system of claim 9,
wherein the screen display comprises a longitudinal view of the body lumen based on the plurality of intraluminal images,
wherein the second marking corresponds to an initial intraluminal image of the plurality of intraluminal images, and
wherein the third marking corresponds to a final intraluminal image of the plurality of intraluminal images.

11. The system of claim 1, wherein the screen display comprises:
a fourth marking disposed along the guidewire in the extraluminal image at the corresponding position of a bookmarked intraluminal image of the plurality of intraluminal images.

12. The system of claim 11, wherein the processor circuit is configured to automatically provide the fourth marking in the extraluminal image in response to the bookmarked intraluminal image being bookmarked.

13. The system of claim 12, wherein the bookmarked intraluminal image is manually bookmarked based on a user input received by the processor circuit.

14. The system of claim 12, wherein the bookmarked intraluminal image is automatically bookmarked by the processor circuit.

15. The system of claim 11, wherein the screen display comprises:
a longitudinal view of the body lumen based on the plurality of intraluminal images; and
a fifth marking at a site in the longitudinal view associated with the corresponding position of the bookmarked intraluminal image.

16. A system, comprising:
an intravascular ultrasound (IVUS) imaging catheter; and
a processor circuit configured for communication with an x-ray imaging device and the IVUS imaging catheter, wherein the processor circuit is configured to:
receive a plurality of x-ray images obtained by the x-ray imaging device during movement of the IVUS imaging catheter along a guidewire within a blood vessel of a patient, wherein the plurality of x-ray images are obtained without a contrast agent within the blood vessel, wherein the plurality of x-ray images show the guidewire and a radiopaque portion of the IVUS imaging catheter;
receive a plurality of IVUS images obtained by the IVUS imaging catheter during the movement of the IVUS imaging catheter;
determine a path of the movement based on corresponding locations of the radiopaque portion in the plurality of x-ray images, wherein a shape of the path matches a shape of the guidewire;
co-register the plurality of IVUS images to corresponding locations along the path such that the plurality of IVUS images are co-registered with corresponding positions along the guidewire; and
output, to a display in communication with the processor circuit, a screen display comprising:
an x-ray image of the plurality of x-ray images;
an IVUS image of the plurality of IVUS images; and
a first marking along the guidewire in the x-ray image representative a corresponding position of the IVUS image.

17. The system of claim 15, wherein the screen display further comprises:
a second marking along the guidewire in the IVUS image representative of a bookmarked IVUS image of the plurality of IVUS images.
